(19) United States
(12) Patent Application Publication
(10) Pub. No.: US 2025/0304916 A1
(43) Pub. Date: Oct. 2, 2025

(54) METHODS AND COMPOSITIONS FOR GENERATING VASCULAR LEPTOMENINGEAL CELLS

(71) Applicant: Trailhead Biosystems Inc., Beachwood, OH (US)

(72) Inventor: Nooshin AMINI, Cleveland, OH (US)

(21) Appl. No.: 19/076,359

(22) Filed: Mar. 11, 2025

Related U.S. Application Data
(60) Provisional application No. 63/563,753, filed on Mar. 11, 2024.

(51) Int. Cl. C12N 5/077

(52) U.S. Cl. CPC C12N 5/0656; C12N 2500/36; C12N 2500/38; C12N 2501/105; C12N 2501/115; C12N 2501/135; C12N 2501/395; C12N 2501/415; C12N 2501/42; C12N 2501/727; C12N 2506/45

(57) ABSTRACT

Methods for generating human vascular leptomeningeal cells (VLMCs) from human oligodendrocyte progenitor cells (OPCs) are provided using chemically-defined culture media that allow for generation of differentiated cells in a 34-day culture protocol. Methods of generating human VLMCs from human pluripotent stem cells using chemically-defined culture media in a 40 day culture protocol are also provided. Culture media, isolated cell populations, and kits are also provided.

### BACKGROUND OF THE INVENTION

The leptomeninges are the two innermost layers of tissue covering the brain and spinal cord, called the arachnoid mater and the pia mater. Leptomeningeal disease (also known as LMD, leptomeningeal metastases, or LM) occurs when an advanced cancer spreads to the cerebrospinal fluid and leptomeninges of the patient. LMD can arise from a variety of malignancies, including breast cancer, lung cancer, and melanoma, and is a late-stage terminal complication. While certain recent treatment modalities have improved survival somewhat, prognosis typically is no more than 3-6 months.

Vascular leptomeningeal cells (VLMC) are unique fibroblast-like cells found at the interface between astrocytes and endothelium covering the brain and spinal cord. In addition to contributing to the protective layer around the central nervous system (CNS), VLMCs play a role in the migration and proliferation of neuronal cells by providing membrane proteins such as laminins and collagens and developmental proteins such as retinoic acid and bone morphogenetic proteins. Another role of leptomeningeal cells in the brain is regulation of the inflammatory response of surrounding cells in the blood brain barrier region. It has been reported that leptomeningeal cells activate astrocytes and microglia by secretion of proinflammatory cytokines such as IL-1β and TNF-α. Thus, it is thought that leptomeningeal cells may transduce peripheral proinflammatory signals to the central anti-inflammatory response through the activation of glial cells in the brain parenchyma.

The understanding of leptomeningeal cell biology and their function in the CNS, as well as their role in LMD, is hampered by a lack of culture differentiation protocols for such cells. In particular, there currently are no differentiation protocols for obtaining VLMCs from pluripotent stem cells or oligodendrocyte progenitor cells. Accordingly, there remains a need for such protocols in the art.

### SUMMARY OF THE INVENTION

This disclosure provides methods of generating human vascular leptomeningeal cells (VLMCs) from oligodendrocyte progenitor cells (OPCs) through a multipotent glial progenitor cell intermediate using chemically-defined culture media. The OPCs can be obtained from pluripotent stem cells, also using chemically-defined culture media. The methods described herein allow for generation of functional VLMCs from OPCs in a two-stage 34-day culture protocol or from pluripotent stem cells in a four-stage 40-day protocol. The culture media described herein comprise small molecule agents that either agonize or antagonize particular signaling pathway activity in the OPCs or pluripotent stem cells such that differentiation along the VLMC lineage is promoted, leading to cellular maturation and expression of VLMC-associated biomarkers. The use of small molecule agents in the culture media allows for precise control of the culture components.

Accordingly, in one aspect, the disclosure pertains to a method of generating human VLMCs from human OPCs, the method comprising:
culturing human OPCs in a first culture media comprising a fibroblast growth factor receptor (FGFR) pathway agonist, a retinoic acid (RA) pathway agonist, a WNT pathway agonist, a NOTCH pathway antagonist, a platelet-derived growth factor receptor (PDGFR) pathway agonist, and an insulin-like growth factor 1 (IGF-1) pathway agonist such that human glial progenitor cells (GPCs) are generated; and
further culturing the GPCs in a second culture media comprising a tropomyosin receptor kinase C (TrkC) pathway agonist, a thyroid hormone receptor (THR) pathway agonist, a protein kinase A (PKA) pathway agonist, and a NOTCH pathway antagonist such that human VLMCs are generated.

In embodiments, human GPCs are generated after six days of culture of the human OPCs in the first culture media.

In embodiments, human VLMCs are generated after 28 days of culture of the human GPCs in the second culture media.

In embodiments, the human GPCs express one or more markers selected from the group consisting of PDGFRA, OLIG2, SOX10, SOX8, NKX2-2, and NG2.

In embodiments, the human VLMCs express one or more markers selected from the group consisting of DCN, LUM, COL1A1, MBP, PRPX1, and MMP2. In embodiments, the human VLMCs also express one or more markers selected from the group consisting of APOE, RSG4, PDGFRA, PDGFRB, NG2, A2B5, CNP, CSPG4, NNAT, CD9, CD146, IGFBP2, MYC, MYT-1, KCNJ8, and OLIG1.

In embodiments, the human OPCs express one or more markers selected from the group consisting of CD9, BCAN, PTPRZ1, and SOX10.

In embodiments, the FGFR pathway agonist is selected from the group consisting of FGF2, SUN11602, FGF1, FGF3, FGF4, FGF5, FGF6, FGF8, FGF10, FGF17, FGF19, FGF20, FGF21, FGF22, FGF23, and combinations thereof. In embodiments, the FGFR pathway agonist is FGF2. In embodiments, FGF2 is present in the first culture media at a concentration of 10 ng/ml.

In embodiments, the RA pathway agonist is selected from the group consisting of TTNPB, retinoic acid (ATRA), EC23, 9-cis-retinoic acid, adapalene, tretinoin, 13-cis retinoic acid (isotretinoin), 4-oxo retinoic acid, WYC-209, DC271, acitretin, arotinoid, AGN205327, LGD1550, Ch55, tazarotene (AGN190168), AM 580, CD2081, BMS 753, tamibarotene, AGN194078, AGN195183, AGN193836, CD2314, CD2019, CD666, C286, BMS 641, AC-55649, AC261066, KCL-286, CD 1530, CD 437, CD2325, BMS 189961, BMS 270394, BMS 961, trifarotene, palovarotene, SR11237, and combinations thereof. In embodiments, the RA pathway agonist is TTNPB. In embodiments, TTNPB is present in the first culture media at a concentration of 50 nM.

In embodiments, the WNT pathway agonist is selected from the group consisting of CHIR99021, CHIR98014, SB 216763, SB 415286, LY2090314, 3F8, A 1070722, AR-A 014418, BIO, BIO-acetoxime, AZD1080, alsterpaullone, indirubin-3-oxime, 1-azakenpaullone, kenpaullone, TC-G 24, TWS 119, AT 7519, KY 19382, AZD2858, CHIR98023, 6-BIO, Cazpaullone, Aloisine A, SB41528, SAR502250, Hymenialdisine, Debromohymenialdisine, Dibromocantherelline, Meridianine A, NSC 693868, IM-12, IMID1, IMID2, VP2.51, VP2.54, BIP-135, JGK-263, MMBO, TCS2002, PF-367, BRD0705, BRD3731, AF3581, TDZD 8, NP 031112, NP00111, NP031115, L803, L803-mts, L807-mts, HMK-32, Palinurin, Tricantin, Manzamine A, BTO, VP0.7, VP1.14, VP1.16, VP3.15, VP3.35, SC100, 6j, LCQFGS01, LCQFGS02, 4-3, 4-4, and combinations thereof. In embodiments, the WNT pathway agonist is CHIR99021. In embodiments, CHIR99021 is present in the first culture media at a concentration of 1 μM.

In embodiments, the NOTCH pathway antagonist is selected from the group consisting of Dibenzazepine (DBZ), GSI-XX, RO4929097, Semagacestat, LY411575, Crenigacestat, DAPT, BMS 906024, Avagacestat, BMS 299897, BMS 433796, BMS 986115, Compound E, Compound W, Compound 18, DFK-167, L-685458, LY900009, MK-0752, MRK 003, MRK 560, PF 3084014, PF 3084014 Hydrobromide, Z-IL-CHO, Begacestat, JLK6, AL101, IMR-1, IMR-1A, CB-103, RIN1, Brontictuzumab, Tarextumab, PF-06650808, FLI-06, Thapsigargin, CAD204520, Tangeretin, Bruceine D, 15D11, Enoticumab, Demcizumab, ABT-165, Navicixizumab, Marimastat, ZLDI-8, and combinations thereof. In embodiments, the NOTCH pathway antagonist is Dibenzazepine (DBZ). In embodiments, DBZ is present in the first culture media at a concentration of 100 nM. In embodiments, DBZ is present in the second culture media at a concentration of 100 nM.

In embodiments, the PDGFR pathway agonist is selected from the group consisting of PDGF-AA, PDGF-BB, PDGF-AB, PDGF-CC, PDGF-DD, PBA2-1c, PMP1, PMP2, and combinations thereof. In embodiments, the PDGFR pathway agonist is PDGF-AA. In embodiments, PDGF-AA is present in the first culture media at a concentration of 10 ng/ml.

In embodiments, the IGF-1 pathway agonist is selected from the group consisting of IGF-1, IGF1-Ado, X10, mecasermin, IGF-2, insulin, Rg5, IGF-1 24-41, IGF-1 30-41, des (1-3) IGF-1, IGF-1 LR3, Demethylasterriquinone B1, and combinations thereof. In embodiments, the IGF-1 pathway agonist is IGF-1. In embodiments, IGF-1 is present in the first culture media at a concentration of 10 ng/ml.

In embodiments, the first culture media further comprises a polyunsaturated fatty acid (PFA). In embodiments, the PFA is selected from the group consisting of linoleic acid, α-linoleic acid (ALA), stearidonic acid (SDA), cicosapentaenoic acid (EPA), docosapentaenoic acid (DPA), docosahexaenoic acid (DHA), γ-linoleic acid (GLA), dihomo-γ-linoleic acid (DGLA), hexadecatrienoic acid (HTA), cicosatrienoic acid (ETE), cicosatetraenoic acid (ETA), heneicosapentaenoic acid (HPA), tetracosapentaenoic acid, tetracosahexaenoic acid, eicosadienoicd acid, arachidonic acid (AA), docosadienoic acid, adrenic acid (AdA), tetracosatetraenoic acid, tetracosapentaenoic acid, conjugated linoleic acid (CLA), conjugated linolenic acid, rumelenic acid, α-parinaric acid, β-parinaric acid, bosseopentaenoic acid, pinolenic acid, sciadonic acid, and combinations thereof. In embodiments, the PFA is linoleic acid. In embodiments, linoleic acid is present in the first culture media at a concentration of 100 μM.

In embodiments, the TrkC pathway agonist is selected from the group consisting of neurotrophin-3 (NT-3), peptidomimetics based on β-turns of NT-3, LM22B 10, GNF 5837, and combinations thereof. In embodiments, the TrkC pathway agonist is NT-3. In embodiments, NT-3 is present in the second culture media at a concentration of 10 ng/ml.

In embodiments, the THR pathway agonist is selected from the group consisting of T3, T4, Tiratricol, Liothyronine, Octinoxate, 3,5-Diiodothyropropinonic acid, Eprotirome, CO23, Resmetirom, Sobetirome, Sob-AM2, ZTA-261, MB-07811, MB-07344, ALG-055009, and combinations thereof. In embodiments, the THR pathway agonist is T3. In embodiments, T3 is present in the second culture media at a concentration of 100 nM.

In embodiments, the PKA pathway agonist is selected from the group consisting of CAMP, Dibutyryl-cAMP, 8-Br-CAMP, CAMPS-Sp, CW 008, Forskolin, 8-CPT-CAMP, Adenosine 3′, 5′-cyclic Monophosphate, N6-Benzoyl-cAMP, Sodium Salt, Adenosine 3′, 5′-cyclic monophosphate sodium salt monohydrate, (S)-Adenosine, cyclic 3′, 5′-(hydrogenphosphorothioate)triethylammonium, Sp-Adenosine 3′,5′-cyclic monophosphorothioate tricthylammonium salt, Sp-5,6-DCI-cBiMPS, 8-Bromoadenosine 3′, 5′-cyclic Monophosphothioate, Sp-Isomer sodium salt, Adenosine 3′, 5′-cyclic Monophosphorothioate, 8-Bromo-CAMP, Sp-Isomer, Sp-8-pCPT-cyclic GMPS Sodium, 8-Bromoadenosine 3′, 5′-cyclic monophosphate, N6-Monobutyryladenosine 3′: 5′-cyclic monophosphate sodium salt, 8-PIP-CAMP, Sp-CAMPS, and combinations thereof. In embodiments, the PKA pathway agonist is cAMP. In embodiments, cAMP is present in the second culture media at a concentration of 1 μM.

In embodiments, the second culture media further comprises a short chain fatty acid (SCFA). In embodiments, the SCFA is selected from the group consisting of propionate (propionic acid), acetate (acetic acid), butyrate (butyric acid), valerate (valeric acid), isobutyrate (isobutyric acid), isovalerate (isovaleric acid), 2-methylbutanoate (2-methylbutyric acid), and combinations thereof. In embodiments, the SCFA is propionate. In embodiments, propionate is present in the second culture media at a concentration of 100 nM.

In another aspect, the disclosure provides a method of generating human VLMCs from human pluripotent stem cells, the method comprising:
culturing human pluripotent stem cells in a culture media comprising an RA pathway agonist, an AKT pathway agonist, a mammalian target of rapamycin (mTOR) pathway agonist, a WNT pathway antagonist, a sonic hedgehog (SHH) pathway agonist, a bone morphogenetic protein (BMP) pathway antagonist, and a protein kinase C (PKC) pathway antagonist for three days such that human pre-oligodendrocyte progenitor cells (pre-OPCs) are generated;
culturing the human pre-OPCs in a culture media comprising an FGFR pathway agonist, an SHH pathway agonist, an AKT pathway antagonist, an AKT pathway agonist, and an mTOR pathway antagonist for three days such that human OPCs are generated;
culturing the human OPCs in a culture media comprising an FGFR pathway agonist, an RA pathway agonist, a WNT pathway agonist, a NOTCH pathway antagonist, a PDGFR pathway agonist, and an IGF-1 pathway agonist (and optionally a PFA) for six days such that human GPCs are generated; and
culturing the human GPCs in a culture media comprising a TrkC pathway agonist, a THR pathway agonist, a PKA pathway agonist, and a NOTCH pathway antagonist (and optionally a SCFA) for 28 days such that human VLMCs are generated.

In yet another aspect, the disclosure pertains to culture media compositions. In embodiments, the disclosure provides a culture media for obtaining human GPCs comprising an FGFR pathway agonist, an RA pathway agonist, a WNT pathway agonist, a NOTCH pathway antagonist, a PDGFR pathway agonist, and an IGF-1 pathway agonist (and optionally a polyunsaturated fatty acid). In embodiments, the disclosure provides a culture media for obtaining human VLMCs comprising a TrkC pathway agonist, a THR pathway agonist, a PKA pathway agonist, and a NOTCH pathway antagonist (and optionally a short chain fatty acid).

In yet other aspects, the disclosure pertains to isolated cell cultures. In embodiments, the disclosure provides an isolated cell culture of human VLMCs, the culture comprising: human VLMCs cultured in a culture media comprising a TrkC pathway agonist, a THR pathway agonist, a PKA pathway agonist, and a NOTCH pathway antagonist (and optionally a short chain fatty acid).

In yet another aspect, the disclosure pertains to isolated VLMCs and cell populations thereof. In embodiments, the disclosure provides an isolated human VLMC, wherein the cell:
(i) is derived from a pluripotent stem cell (e.g., ES cell or iPSC) or OPC;
(ii) has a fibroblast-like morphology;
(iii) expresses at least one VLMC marker selected from the group consisting of DCN, LUM, COL1A1, MBP, PRPX1, and MMP2; and
(iv) expresses at least one additional marker selected from the group consisting of APOE, RSG4, PDGFRA, PDGFRB, NG2, A2B5, CNP, CSPG4, NNAT, CD9, CD146, IGFBP2, MYC, MYT-1, KCNJ8, and OLIG1.

In embodiments, the VLMC expresses two, three, four, five, or all six VLMC markers selected from DCN, LUM, COL1A1, MBP, PRPX1, and MMP2.

In embodiments, the VLMC secretes IL-1β upon inflammatory stimulation. In embodiments, inflammatory stimulation comprises culture with lipopolysaccharide (LPS).

In another aspect, the disclosure provides a cultured cell population comprising at least 1×10⁶ (e.g., at least 1×10⁷, 1×10⁸, 1×10⁹, or more) of the human VLMCs described herein. In embodiments, the cultured cell population is in a suspension culture. In embodiments, the cultured cell population is in an adherent culture.

In an embodiment of any of the foregoing aspects, the FGFR pathway agonist is a FGFR agonist.

In an embodiment of any of the foregoing aspects, the RA pathway agonist is an RA agonist.

In an embodiment of any of the foregoing aspects, the NOTCH pathway antagonist is a NOTCH antagonist.

In an embodiment of any of the foregoing aspects, the WNT pathway agonist is a WNT agonist. In an embodiment of any of the foregoing aspects, the WNT pathway antagonist is a WNT antagonist.

In an embodiment of any of the foregoing aspects, the PDGFR pathway agonist is a PDGFR agonist.

In an embodiment of any of the foregoing aspects, the IGF-1 pathway agonist is an IGF-1 agonist.

In an embodiment of any of the foregoing aspects, the TrkC pathway agonist is a TrkC agonist.

In an embodiment of any of the foregoing aspects, the THR pathway agonist is a THR agonist.

In an embodiment of any of the foregoing aspects, the PKA pathway agonist is a PKA agonist.

In an embodiment of any of the foregoing aspects, the Akt pathway agonist is an Akt agonist. In an embodiment of any of the foregoing aspects, the Akt pathway antagonist is an Akt antagonist.

In an embodiment of any of the foregoing aspects, the mTOR pathway agonist is an mTOR agonist. In an embodiment of any of the foregoing aspects, the mTOR pathway antagonist is an mTOR antagonist.

In an embodiment of any of the foregoing aspects, the SHH pathway agonist is a SHH agonist.

In an embodiment of any of the foregoing aspects, the BMP pathway antagonist is a BMP antagonist.

In an embodiment of any of the foregoing aspects, the PKC pathway antagonist is a PKC antagonist.

Other features and advantages of the invention will be apparent from the following detailed description and claims.

### BRIEF DESCRIPTION OF THE DRAWINGS

FIG. 1 shows the results from an HD-DoE model of an 8-factor experiment optimized for maximum expression of SOX10. The upper section of the model shows the prediction of expression level of pre-selected 53 genes when optimized for SOX10. The lower section of the model shows the effectors that were tested in this model and their contribution to maximum expression of SOX10. The value column refers to the required concentration of each effector to mimic the model.

FIG. 2 shows the results from an HD-DoE model of an 8-factor experiment optimized for maximum expression of PDGFRA. This condition highlights the effector Purmorphamine with a factor contribution of 20.6 and IGF-1 with a factor contribution of 11.22 as two important inputs for maximal expression of PDGFRA.

FIG. 3 shows the results from an HD-DoE model of an 8-factor experiment optimized for maximum expression of SOX8. This condition highlights the effector, IGF-1 with a factor contribution of 15.6 and MHY1485 with a factor contribution of 13.2 as two important inputs for maximal expression of SOX8.

FIG. 4 shows the dynamic profile of expression levels of NKX2-2, OLIG2, OLIG1, SOX10, SOX8 and PDGFRA relative to concentration of 8 effectors. The positive effect of TTNPB, CHIR99021 and FGF2 on expression of SOX10 when the model is optimized for its maximal expression and their factor contributions are shown by slope of the plots for each effector.

FIG. 5 shows the results from an HD-DoE model of a 12-factor experiment optimized for maximum expression of OLIG1 and NKX2-2 simultaneously. This condition highlights the effector, PDGF-AA with a factor contribution of 21 and GSI-XX (DBZ) with a factor contribution of 13.2 as two important inputs for their maximal expression.

FIG. 6 shows the results from an HD-DoE model of an 8-factor experiment optimized for maximum expression of MBP. This condition highlights the effector T3 with a factor contribution of 18.1 and GSI-XX (DBZ) with a factor contribution of 18.1 as two important inputs for maximal expression of MBP.

FIG. 7 shows a photograph of phase image of differentiated cells in adherent culture on day 40 of differentiation. The white arrow points to the fibroblast-like morphology of cells in the majority of culture.

FIGS. 8A and 8B are plots showing result of flow cytometry assays performed on iPSC-derived VLMCs on day 7 (FIG. 8A) and day 14 (FIG. 8B) of stage 4 of differentiation. The percentage of detected markers in VLMC and pre-OL cells was calculated using FlowJo software after gating over singlets-doublets and live-dead populations. Single color and multi color controls minus one antibody (FMO) were used as positive and negative controls for each panel.

FIGS. 9A-9D show bulk RNA-seq result of iPSC-derived VLMCs on day 7 and day 28 of stage 4 of differentiation. FIG. 9A shows the heatmap of normalized log 2 count of selected set of genes on day 28 in VLMC and pre-OL cells. FIG. 9B shows the normalized log 2 count of VLMC genes on day 7 in both populations. FIG. 9C shows the normalized log 2 count of VLMC genes on day 28 in both populations. Significant differences were calculated using one-way ANOVA in Prism GraphPad software. FIG. 9D is a schematic depiction of multipotential glial progenitors differentiating to either pre-OL cells or VLMCs.

FIG. 10 shows photographs of immunofluorescent images of iPSC derived VLMCs at end of stage 4 treatment (day 40). Cells express VLMC markers LUM, DCN, COL1A1 and PDGFRa.

FIG. 11A shows a photograph of phase images of VLMCs on day 14 of stage 4 at hour 0 and after 4 hours of stimulation with 10 ng/ml LPS. FIG. 11B shows a plot of IL-1b secretion from cells over 24 hours in cell culture media+LPS. Analysis was done using calibration curve obtained from ELISA of standard samples provided by manufacturers.

FIG. 12 shows photographs of phase images of VLMCs on the last day of suspension culture, one day after dissociation and plating onto coated plates and after weekly split to control cell density.

FIG. 13 shows photographs of fluorescence images of hiPSC-derived VLMCs at the end of stage 4 in suspension culture. Cells were stained with VLMC biomarkers including COL1A1, DCN, LUM, NG2, IL-33, VTN, a-SMA and KI67. At this stage cells were positive for all the expected markers. The presence of KI67 in a fraction of culture showed a minimal population of cells were still proliferative.

FIG. 14A shows the results of a gene profile analysis of iPSC-derived VLMCs on day 14 and 28 of stage 4 in suspension culture as measured by qPCR. The fold change, relative to expression of genes to GAPDH (housekeeping gene), on both timepoints confirms increasing expression of VLMC genes while expression of neuronal markers TUBB3 and NES was decreased. FIG. 14B shows results of a protein expression analysis of VLCM markers measured by flow cytometry on day 10 and 28 of stage of differentiation confirms more than 50% of generated cells have VLMC identity.

FIG. 15 shows the results of Bulk RNA-seq analysis of iPSC-derived VLMCs on day 1 and day 14 of stage 4 of differentiation in suspension culture. The heatmap depicts normalized log 2 count of selected set of genes on both timepoints categorized by cell type specific genes. An increasing trend in VLMC genes was observed as cells differentiate in stage 4 media.

FIG. 16 shows photographs of fluorescence images of hiPSC-derived VLMCs on a second iPSC line at the end of stage 4. Cells were stained with VLMC biomarkers including COL1A1, DCN, LUM, NG2, and KI67. At this stage cells were positive for all the expected markers. The presence of KI67 in a fraction of culture showed a minimal population of cells were still proliferative.

FIG. 17 shows results of flow cytometry assays performed on iPSC-derived VLMCs using a second iPSC line on the last day of differentiation. More than 80% of generated cells expressed target VLMC markers. The percentage of detected markers was calculated using FlowJo software.

### DETAILED DESCRIPTION OF THE INVENTION

Described herein are methodologies and compositions that allow for the generation of vascular leptomeningeal cells (VLMCs) from human oligodendrocyte progenitor cells (OPCs) or human pluripotent stem cells (PSCs) under chemically-defined culture conditions using a small molecule based approach. As described in Example 1, a High-Dimensional Design of Experiments (HD-DoE) approach was used to simultaneously test multiple process inputs (e.g., small molecule agonists or antagonists) on output responses, such as gene expression. These experiments allowed for the identification of a two-stage culture protocol using chemically-defined culture media, comprising agonists and/or antagonists of particular signaling pathways, that is sufficient to generate multipotential glial progenitor cells (GPCs) from OPCs in six days and VLMCs from the GPCs by further culture for 28 days, thereby providing VLMCs from OPCs in 34 days. Starting from PSCs, an additional two-stage, six-day protocol for generating OPCs from PSCs is conducted, thereby providing VLMCs from PSCs in 40 days.

As described in Example 2, the phenotype of the differentiated VLMCs was validated by RNA sequence analysis, immunocytochemistry, and flow cytometry. Moreover, the VLMCs were functionally validated by demonstrating IL-1β secretion upon lipopolysaccharide (LPS) induction (see Example 3). Furthermore, the culture protocol described herein has been validated for both adherent and suspension cultures (see Examples 2 and 4) and has been validated using two different induced PSC (iPSC) lines as the starting cells (see Examples 2 and 5).

Various aspects of the invention are described in further detail in the following subsections.

### I. Cells

In embodiments, the starting cells used in the cultures of the disclosure are human pluripotent stem cells. As used herein, the terms “human pluripotent stem cell” and “hPSC” refer to a human stem cell that has the capacity to differentiate into a variety of different cell types. The term “pluripotent” as used herein refers to a cell with the capacity, under different conditions, to differentiate to cell types characteristic of all three germ cell layers (endoderm, mesoderm, and ectoderm). Pluripotent cells are characterized primarily by their ability to differentiate to all three germ layers, for example, using a nude mouse and teratomas formation assay. Pluripotency can also be evidenced by the expression of embryonic stem (ES) cell markers, although the preferred test for pluripotency is the demonstration of the capacity to differentiate into cells of each of the three germ layers.

Human pluripotent stem cells include, for example, induced pluripotent stem cells (iPSC) and human embryonic stem cells, such as ES cell lines. Non-limiting examples of iPSCs are 19-11-1, 19-9-7, or 6-9-9 cells (e.g., as described in Yu, J. et al. (2009) Science 324:797-801) and Human iPSCs 771-3G, 802-3G, SK001.1, SK004.2, SK002.1, SK005.3, SK003.2, SK006.4, and BL003 (commercially available from Reprocell StemRNA). Non-limiting examples of human embryonic stem cell lines are ES03 cells (WiCell Research Institute) and H9 cells (Thomson, J. A. et al. (1998) Science 282:1145-1147). Human pluripotent stem cells (PSCs) express cellular markers that can be used to identify cells as being PSCs. Non-limiting examples of pluripotent stem cell markers are TRA-1-60, TRA-1-81, TRA-2-54, SSEA1, SSEA3, SSEA4, CD9, CD24, OCT3, OCT4, NANOG, and/or SOX2.

In other embodiments, the starting cells used in the cultures of the disclosure are human oligodendrocyte progenitor cells (OPCs). As used herein, the terms “oligodendrocyte progenitor cell” and “OPC” refer to a neuronal progenitor cell that expresses the cellular markers OLIG2 and NKX2.2, as well as PDGFRa. An OPC may express additional markers, non-limiting example of which are SOX10 (neural crest marker), OTX2 (anterior neuroectoderm biomarker), FEZF2 (anterior ectoderm biomarker), and/or OLIG1.

In embodiments, the OPCs are obtained from PSCs according to a two-stage protocol that generates pre-oligodendrocyte progenitor cells (pre-OPCs) as an intermediate cell type. As used herein, the terms “pre-oligodendrocyte progenitor cell” and “pre-OPC” refer to a stem cell-derived progenitor cell that expresses the cellular markers OLIG2 and NKX2.2. A pre-OPC may express additional markers, including but not limited to: OTX2 (anterior neuroectoderm biomarker), FEZF2 (anterior ectoderm biomarker), and/or OLIG1.

In embodiments, the human OPCs express one or more markers (e.g., one, two, three, or all four markers) selected from the group consisting of CD9, BCAN, PTPRZ1, and SOX10.

The starting cells are subjected to culture conditions, as described herein, that induce cellular differentiation. As used herein, the term “differentiation” refers to the development of a cell from a more primitive stage towards a more mature (i.e., less primitive) cell, typically exhibiting phenotypic features of commitment to a particular cellular lineage.

Methods of differentiating pluripotent stem cells into OPCs, through a pre-OPC intermediate, have been described in the art that are suitable for use in combination with the VLMC protocols described herein. For example, a 3-day protocol for generating pre-OPCs from pluripotent stem cells is described in US 2022/0315891, and a 3-day protocol for generating OPCs from pre-OPCs is described in US 2024/0043798, the entire contents of both of which are expressly incorporated herein by reference.

In embodiments, pre-OPCs are generated from pluripotent stem cells (e.g., iPSCs or ES cells) by culturing the pluripotent stem cells for three days in a culture media comprising a retinoic acid (RA) pathway agonist, an Akt pathway agonist and a mammalian target of rapamycin (mTOR) pathway agonist such that pre-OPCs are generated. In embodiments, the culture media further comprises a WNT pathway antagonist. In embodiments, the culture media further comprises a sonic hedgehog (SHH) pathway agonist. In embodiments, the culture media further comprises a bone morphogenetic protein (BMP) pathway antagonist. In embodiments, the culture media further comprises a protein kinase C (PKC) pathway antagonist. Suitable culture media components are described further in Subsection II. A non-limiting example of a culture media for differentiating PSCs to pre-OPCs, referred to herein as the Stage 1 culture media, is shown below in Table 1:

TABLE 1
Stage 1 culture media for differentiating PSCs to pre-OPCs

| Effectors | Role | Concentration | |
|---|---|---|---|
| TTNPB | RA pathway agonist | 50 | nM |
| SC79 | AKT pathway agonist | 1 | μM |
| MHY1485 | mTOR pathway agonist | 1 | μM |
| XAV939 | WNT pathway antagonist | 100 | nM |
| Purmorphamine | SHH pathway agonist | 500 | nM |
| LDN193189 | BMP pathway antagonist | 250 | nM |
| Go6983 | PKC pathway antagonist | 110 | nM |

In embodiments, OPCs are generated from pre-OPCs by culturing the pre-OPCs for three days in a culture media comprising a fibroblast growth factor receptor (FGFR) pathway agonist, an SHH pathway agonist, an AKT pathway antagonist, an AKT pathway agonist, and an mTOR pathway antagonist such that OPCs are generated. Suitable culture media components are described further in Subsection II. A non-limiting example of a culture media for differentiating pre-OPCs to OPCs, referred to herein as the Stage 2 culture media, is shown below in Table 2:

TABLE 2
Stage 2 culture media for differentiating pre-OPCs to OPCs

| Effectors | Role | Concentration | |
|---|---|---|---|
| FGF2 | FGFR pathway agonist | 10 | ng/ml |
| Purmorphamine | SHH pathway agonist | 500 | nM |
| MK2206 | AKT pathway antagonist | 125 | nM |
| SC79 | AKT pathway agonist | 2 | μM |
| AZD3147 | mTOR pathway antagonist | 15 | nM |

The GPCs and VLMCs generated by the methods of the disclosure can be characterized by, for example, morphology, gene expression, marker expression (e.g., surface marker expression, genetic marker expression), and/or functional activity.

For marker expression, as used herein a population of cells is considered “positive” for the marker if at least 15% of the cells in the population express the marker. In embodiments, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of cells in a cell population express a marker of interest.

In embodiments, the cells have a fibroblast-like morphology. Cells described as having a “fibroblast-like morphology” refer to cells that are bipolar or multipolar, have a flattened, elongated, or spindle-like shape and can grow attached to a substrate (although the cells may also be capable of growth in suspension).

In embodiments, the cells express one or more leptomeningeal markers. In embodiments, the cells also express one or more markers shared with glial or pericyte cells. In embodiments, the cells also express one or more markers shared with oligodendrocytes.

In embodiments, the cells express at least one, at least two, or all three leptomeningeal markers selected from DCN, LUM, and COL1A1. In embodiments, the cells also express one or more (e.g., two, three, four, five, six) leptomeningeal markers selected from CRABP2, APOE, RSG4, S100a6, MBP, and MMP2.

In embodiments, the human GPCs (e.g., generated after six days of culture of OPCs in the stage 3 media described herein) express one or more markers selected from the group consisting of PDGFRA, OLIG2, SOX 10, SOX8, NKX2-2, and NG2. In embodiments, the cells express two, three, four, five, or all six of the markers selected from PDGFRA, OLIG2, SOX 10, SOX8, NKX2-2, and NG2.

In embodiments, the human VLMCs express one or more VLMC markers selected from the group consisting of DCN, LUM, COL1A1, MBP, PRPX1, and MMP2. In embodiments, the VLMCs express two, three, four, five, or all six of the markers selected from DCN, LUM, COL1A1, MBP, PRPX1, and MMP2.

In embodiments, the human VLMCs also express one or more markers selected from the group consisting of APOE, RSG4, PDGFRA, PDGFRB, NG2, A2B5, CNP, CSPG4, NNAT, CD9, CD146, IGFBP2, MYC, MYT-1, KCNJ8, and OLIG1. In embodiments, the VLMCs express two, three, four, five, six, seven, eight, nine, ten, eleven, twelve, thirteen, fourteen, fifteen, or sixteen of the markers selected from APOE, RSG4, PDGFRA, PDGFRB, NG2, A2B5, CNP, CSPG4, NNAT, CD9, CD146, IGFBP2, MYC, MYT-1, KCNJ8, and OLIG1.

In embodiments, the cells express one or more (e.g., two, three, four, five, six, seven, eight, nine, ten, eleven) markers shared with glial or pericyte cells, non-limiting examples of which are OLIG2, SOX10, NG2, CD146, CD44, PDGFB, KCNJ8, CNP, A2B5, CD9, and 04.

In embodiments, the cells express one or more (e.g., two, three, four, five, six, seven, eight, nine, ten, eleven) markers shared with oligodendrocytes, non-limiting examples of which are PDGFRA, NG2, OLIG1, CSPG4, SSP1, and PRRX1.

In embodiments, the cells have the functional activity of expressing one or more pro-inflammatory cytokines upon inflammatory activation (e.g., stimulation with LPS). For example, in embodiments, the proinflammatory cytokine is IL-1β or TNF-α. Suitable assays for measuring pro-inflammatory cytokine expression are well-established in the art, such as ELISA assays (see e.g., Example 3).

### II. Culture Media Components

The methods of the disclosure for generating GPCs and VLMCs comprise culturing oligodendrocyte progenitor cells in a culture media comprising specific agonist and/or antagonists of cellular signaling pathways. In embodiments, the culture media lacks serum. In embodiments, the culture media lacks serum or other exogenously-added growth factors (other than the agents included in the Stage 1, 2, 3, or 4 culture media recipes).

As described in Example 1, a culture media comprising an FGFR pathway agonist, a retinoic acid (RA) pathway agonist, a WNT pathway agonist, a NOTCH pathway antagonist, a platelet-derived growth factor receptor (PDGFR) pathway agonist, an insulin-like growth factor 1 (IGF-1) pathway agonist, and (optionally) a polyunsaturated fatty acid was sufficient to generate multipotential GPCs from OPCs in six days. This is referred to as the Stage 3 culture media, an exemplary embodiment of which is shown in Table 3. As further described in Example 1, a culture media comprising a tropomyosin receptor kinase C (TrkC) pathway agonist, a thyroid hormone receptor (THR) pathway agonist, a protein kinase A (PKA) pathway agonist, a NOTCH pathway antagonist, and (optionally) a short chain fatty acid was sufficient to generate VLMCs from GPCs in 28 days. This is referred to as the Stage 4 culture media, an exemplary embodiment of which is shown in Table 4. The Stage 3 and Stage 4 culture media and protocols can be combined with the Stage 1 and 2 culture media and protocols described in Subsection I, with exemplary embodiments shown in Tables 1 and 2, to provide a 4-stage, 40-day protocol for generating VLMCs from PSCs.

As used herein, an “agonist” of a cellular signaling pathway is intended to refer to an agent that stimulates (upregulates) the cellular signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with component(s) of the signaling pathway.

As used herein, an “antagonist” of a cellular signaling pathway is intended to refer to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with component(s) of the signaling pathway.

Agonists of the FGFR pathway include agents, molecules, compounds, or substances capable of stimulating (upregulating) the fibroblast growth factor receptor signaling pathway, which biologically is activated by binding of an FGF to an FGFR. In some embodiments, an FGFR pathway agonist binds to an FGFR and stimulates or increases the activity of or through the FGFR. In some embodiments, an FGFR pathway agonist is capable of (i) increasing the expression of FGFR, (ii) increasing FGF-induced phosphorylation of ERK, or (iii) increasing FGF-induced phosphorylation of AKT. In some embodiments, the FGFR pathway agonist is a naturally occurring ligand. In some embodiments, the naturally occurring ligand of FGFR pathway is FGF2, FGF1, FGF3, FGF4, FGF5, FGF6, FGF8, FGF10, FGF17, FGF19, FGF20, FGF21, FGF22, or FGF23. In some embodiments, the FGFR pathway agonist is a ligand mimetic. In some embodiments, the ligand mimetic is SUN11602. In some embodiments, the FGFR pathway agonist is selected from the group consisting of FGF2, SUN11602, FGF1, FGF3, FGF4, FGF5, FGF6, FGF8, FGF10, FGF17, FGF19, FGF20, FGF21, FGF22, and FGF23 or combinations thereof. In one embodiment, the FGFR pathway agonist is present in the culture media at a concentration within a range of 1-20 ng/ml, 5-15 ng/ml, 7.5-12.5 ng/ml, 9-11 ng/ml or at a concentration of 10 ng/ml. In one embodiment, the FGFR pathway agonist is FGF2. In one embodiment, the FGFR pathway agonist is FGF2, which is present in the Stage 3 culture media at a concentration within a range of 1-20 ng/ml, 5-15 ng/ml, 7.5-12.5 ng/ml, or 9-11 ng/ml. In one embodiment, the FGFR pathway agonist is FGF2, which is present in the Stage 3 culture media at a concentration of 10 ng/ml.

Agonists of the RA pathway include agents, molecules, compounds, or substances capable of stimulation of a retinoic acid receptor (RAR) that is activated by both all-trans RA and 9-cis RA. There are three RARs: RARα, RARβ, and RARγ, which are encoded by the RARA, RARB, RARG genes, respectively. Different RA analogs have been synthesized that can activate the RA pathway. In some examples, an RA pathway agonist is capable of increasing RAR mRNA levels. In some embodiments, the RA pathway agonist binds one or more RAR subtypes and stimulates or increases signaling through or activity of the one or more RAR subtypes. In some embodiments, the RA pathway agonist is a pan-RAR agonist. In some embodiments, the pan-RAR agonist is TTNPB, retinoic acid (ATRA), EC23, 9-cis-retinoic acid, adapalene, tretinoin, 13-cis retinoic acid (isotretinoin), 4-oxo retinoic acid, WYC-209, DC271, acitretin, arotinoid, AGN205327, or LGD1550. In some embodiments, the RA pathway agonist is an RARα/β agonist. In some embodiments, the RARα/β agonist is Ch55. In some embodiments, the RA pathway agonist is an RARβ/γ agonist. In some embodiments, the RARβ/γ agonist is tazarotene (AGN190168). In some embodiments, the RA pathway agonist is selective for RARα. In some embodiments, the RARα-selective agonist is AM580 (CD336), CD2081, BMS 753, tamibarotene, AGN194078, AGN195183, or AGN193836. In some embodiments, the RA pathway agonist is selective for RARβ. In some embodiments, the RARβ-selective agonist is CD2314, CD2019, CD666, C286, BMS 641, AC-55649, AC261066, or KCL-286. In some embodiments, the RA pathway agonist is selective for RARγ. In some embodiments, the RARγ-selective agonist is CD1530, CD437, CD2325, BMS 189961, BMS 270394, BMS 961, trifarotene, or palovarotene. In some embodiments, the RA pathway agonist is a retinoid X receptor (RXR) agonist. In some embodiments, the RXR agonist is SR11237.

Accordingly, in some embodiments, the RA pathway agonist is selected from the group consisting of TTNPB, retinoic acid (ATRA), EC23, 9-cis-retinoic acid, adapalene, tretinoin, 13-cis retinoic acid (isotretinoin), 4-oxo retinoic acid, WYC-209, DC271, acitretin, arotinoid, AGN205327, LGD1550, Ch55, tazarotene (AGN190168), AM 580, CD2081, BMS 753, tamibarotene, AGN194078, AGN195183, AGN193836, CD2314, CD2019, CD666, C286, BMS 641, AC-55649, AC261066, KCL-286, CD 1530, CD 437, CD2325, BMS 189961, BMS 270394, BMS 961, trifarotene, palovarotene, SR11237, or combinations thereof. In one embodiment, the RA pathway agonist is present in the culture media at a concentration within a range of 10-100 nM, 20-80 nM, 25-75 nM, or 40-60 nM or at a concentration of 50 nM. In one embodiment, the RA pathway agonist is TTNPB. In one embodiment, the RA pathway agonist is TTNPB, which is present in the Stage 3 culture media at a concentration within a range of 10-100 nM, 20-80 nM, 25-75 nM, or 40-60 nM. In one embodiment, the RA pathway agonist is TTNPB, which is present in the Stage 3 culture media at a concentration of 50 nM.

Agonists of the WNT pathway include agents, molecules, compounds, or substances capable of stimulating (upregulating) the canonical Wnt/β-catenin signaling pathway, which biologically is activated by binding of a Wnt-protein ligand to a Frizzled family receptor. In some embodiments, a WNT pathway agonist is a glycogen synthase kinase 3β (Gsk3β) inhibitor. In some examples, a WNT pathway agonist is capable of (i) decreasing phosphorylation of GSK3β, (ii) decreasing phosphorylation of β-catenin, (iii) increasing expression of β-catenin, (iv) decreasing phosphorylation of PTEN, (v) decreasing phosphorylation of mTOR, or (vi) decreasing phosphorylation of AKT. In some embodiments, the WNT pathway agonist binds a GSK3β enzyme and inhibits or decreases signaling through or activity of the GSK3β enzyme. In some embodiments, the WNT pathway agonist is an ATP-competitive GSK3β inhibitor. In some embodiments, the ATP-competitive GSK3β inhibitor is CHIR99021, CHIR98014, SB 216763, SB 415286, LY2090314, 3F8, A 1070722, AR-A014418, BIO, BIO-acetoxime, AZD1080, alsterpaullone, indirubin-3′-oxime, 1-azakenpaullone, kenpaullone, TC-G 24, TWS119, AT7519, KY19382, AZD2858, CHIR98023, 6-BIO, Cazpaullone, Aloisine A, SB41528, SAR502250, Hymenialdisine, Debromohymenialdisine, Dibromocantherelline, Meridianine A, NSC 693868, IM-12, IMID1, IMID2, VP2.51, VP2.54, BIP-135, JGK-263, MMBO, TCS2002, PF-367, BRD0705, BRD3731, or AF3581. In some embodiments, the WNT pathway agonist is a non-ATP-competitive GSK3β inhibitor. In some embodiments, the non-ATP-competitive GSK3β inhibitor is TDZD-8, NP031112, NP00111, NP031115, L803, L803-mts, L807-mts, HMK-32, Palinurin, Tricantin, Manzamine A, BTO, VP0.7, VP1.14, VP1.16, VP3.15, VP3.35, SC100, 6j, LCQFGS01, LCQFGS02, 4-3, or 4-4.

In some embodiments, the WNT pathway agonist is selected from the group consisting of CHIR99021, CHIR98014, SB 216763, SB 415286, LY2090314, 3F8, A 1070722, AR-A 014418, BIO, BIO-acetoxime, AZD1080, alsterpaullone, indirubin-3-oxime, 1-azakenpaullone, kenpaullone, TC-G 24, TWS 119, AT 7519, KY 19382, AZD2858, CHIR98023, 6-BIO, Cazpaullone, Aloisine A, SB41528, SAR502250, Hymenialdisine, Debromohymenialdisine, Dibromocantherelline, Meridianine A, NSC 693868, IM-12, IMID1, IMID2, VP2.51, VP2.54, BIP-135, JGK-263, MMBO, TCS2002, PF-367, BRD0705, BRD3731, AF3581, TDZD 8, NP 031112, NP00111, NP031115, L803, L803-mts, L807-mts, HMK-32, Palinurin, Tricantin, Manzamine A, BTO, VP0.7, VP1.14, VP1.16, VP3.15, VP3.35, SC100, 6j, LCQFGS01, LCQFGS02, 4-3, 4-4, and combinations thereof. In one embodiment, the WNT pathway agonist is present in the culture media at a concentration within a range of 0.3-3.0 μM, 0.5-2.0 μM, 0.75-1.5 μM, or 0.9-1.1 μM, or at a concentration of 1.0 μM. In one embodiment, the WNT pathway agonist is CHIR99021. In one embodiment, the WNT pathway agonist is CHIR99021, which is present in the Stage 3 culture media at a concentration within a range of 0.3-3.0 μM, 0.5-2.0 M, 0.75-1.5 μM or 0.9-1.1 μM. In one embodiment, the WNT pathway agonist is CHIR99021, which is present in the Stage 3 culture media at a concentration of 1.0 μM.

Antagonists of the NOTCH pathway include agents, molecules, compounds, or substances capable of inhibiting (downregulating) signaling or activity of the NOTCH transcription factor. In some embodiments, the NOTCH pathway antagonist inhibits or decreases signaling through or activity of a Notch receptor (e.g., NOTCH1, NOTCH2, NOTCH3, and NOTCH4). In some examples, a Notch antagonist is capable of (i) reducing cell surface expression of Notch receptors, (ii) reducing expression of Notch Intracellular Domain (NICD) induced by Delta-like 4 (DLL4), (iii) reducing expression of NOTCH1, or (iv) reducing the expression of HES-1 induced by DLL4. In some embodiments, the Notch antagonist is an inhibitor of membrane trafficking of Notch receptors. In some embodiments, the inhibitor of membrane trafficking of Notch receptors is FLI-06, Thapsigargin, or CAD204520. In some embodiments, the Notch antagonist is an agent targeting the ligands of Notch receptors. In some embodiments, the agent targeting the ligands of Notch receptors is Tangeretin, Bruceine D, 15D11, Enoticumab, Demcizumab, ABT-165, or Navicixizumab. In some embodiments, the Notch antagonist is an inhibitor of A Disintegrin and Metalloproteases (ADAMs). In some embodiments, the inhibitor of ADAMs is Marimastat or ZLDI-8. In some embodiments, the Notch antagonist is a γ-secreatase inhibitor (GSI). In some embodiments, the GSI is Dibenzazepine (DBZ), GSI-XX, RO4929097, Semagacestat (LY 450139), LY411575, Crenigacestat (LY 3039478), DAPT, BMS 906024, Avagacestat, BMS 299897, BMS 433796, BMS 986115, Compound E, Compound W, Compound 18, DFK-167, L-685458, LY900009, MK-0752, MRK 003, MRK 560, PF 3084014, PF 3084014 Hydrobromide, Z-IL-CHO, Begacestat, JLK6, or AL101. In some embodiments, the Notch antagonist is a transcription blocker. In some embodiments, the transcription blocker is IMR-1, IMR-1A, CB-103, or RIN1. In some embodiments, the Notch antagonist binds a Notch receptor and inhibits or decreases signaling through or activity of the Notch receptor. In some embodiments, the Notch antagonist is an antibody targeting one or more types of Notch receptors. In some embodiments, the antibody targeting one or more types of Notch receptors is Brontictuzumab, Tarextumab, or PF-06650808.

In some embodiments, the NOTCH pathway antagonist is selected from the group consisting of Dibenzazepine (DBZ), GSI-XX, RO4929097, Semagacestat, LY411575, Crenigacestat, DAPT, BMS 906024, Avagacestat, BMS 299897, BMS 433796, BMS 986115, Compound E, Compound W, Compound 18, DFK-167, L-685458, LY900009, MK-0752, MRK 003, MRK 560, PF 3084014, PF 3084014 Hydrobromide, Z-IL-CHO, Begacestat, JLK6, AL101, IMR-1, IMR-1A, CB-103, RINI, Brontictuzumab, Tarextumab, PF-06650808, FLI-06, Thapsigargin, CAD204520, Tangeretin, Bruceine D, 15D11, Enoticumab, Demcizumab, ABT-165, Navicixizumab, Marimastat, ZLDI-8, and combinations thereof. In one embodiment, the NOTCH pathway antagonist is present in the culture media at a concentration within a range of 25-200 nM, 50-150 nM, or 75-125 nM or at a concentration of 100 nM. In one embodiment, the NOTCH pathway antagonist is Dibenzazepine (DBZ). In one embodiment, the NOTCH pathway antagonist is DBZ, which is present in the Stage 3 culture media at a concentration of 25-200 nM, 50-150 nM, or 75-125 nM. In one embodiment, the NOTCH pathway antagonist is DBZ, which is present in the Stage 3 culture media at a concentration of 100 nM.

Agonists of the PDGFR (platelet-derived growth factor receptor) pathway include agents, molecules, compounds, or substances capable of stimulating (activating) signaling through the PDGFR pathway. In some embodiments, a PDGFR pathway agonist binds to a PDGFR (e.g., PDGFRαα, PDGFRαβ, or PDGFRββ) and stimulates or increases the activity of or through the PDGFR. In some examples, a PDGFR pathway agonist is capable of (i) increasing phosphorylation of PDGFRs, (ii) increasing phosphorylation of AKT, or (iii) increasing phosphorylation of ERK. In some embodiments, the PDGFR agonist is a ligand. In some embodiments, the ligand of a PDGFR is PDGF-AA, PDGF-BB, PDGF-AB, PDGF-CC, or PDGF-DD. In some embodiments, the PDGFR agonist is a ligand mimetic. In some embodiments, the ligand mimetic is PBA2-1c, PMP1, or PMP2. In some embodiments, the PDGFR pathway agonist is selected from the group consisting of PDGF-AA, PDGF-BB, PDGF-AB, PDGF-CC, PDGF-DD, PBA2-1c, PMP1, PMP2, and combinations thereof. In one embodiment, the PDGFR pathway agonist is present in the culture media at a concentration within a range of 2-20 ng/ml, 5-15 ng/ml, or 7.5-12.5 ng/ml. In one embodiment, the PDGFR pathway agonist is PDGF-AA, which is present in the Stage 3 culture media at a concentration of 2-20 ng/ml, 5-15 ng/ml, or 7.5-12.5 ng/ml. In one embodiment, the PDGFR pathway agonist is PDGF-AA, which is present in the Stage 3 culture media at a concentration of 10 ng/ml.

Agonists of the IGF-1 (insulin-like growth factor 1) pathway include agents, molecules, compounds, or substances capable of stimulating (activating) signaling through the IGF-1 pathway. In some embodiments, an IGF-1 pathway agonist binds to an IGF-1 receptor and stimulates or increases the activity of or through the IGF-1 receptor. In some examples, an IGF-1 pathway agonist is capable of (i) increasing IGF-induced phosphorylation of AKT or (ii) increasing IGF-induced phosphorylation of ERK. In some embodiments, the IGF-1 pathway agonist is selected from the group consisting of IGF-1, IGF1-Ado, X10, mecasermin, IGF-2, insulin, Rg5, IGF-1 24-41, IGF-1 30-41, des (1-3) IGF-1, IGF-1 LR3, Demethylasterriquinone B1, and combinations thereof. In one embodiment, the IGF-1 pathway agonist is present in the culture media at a concentration within a range of 2-20 ng/ml, 5-15 ng/ml, or 7.5-12.5 ng/ml or at a concentration of 10 ng/ml. In one embodiment, the IGF-1 pathway agonist is IGF-1. In one embodiment, the IGF-1 pathway agonist is IGF-1, which is present in the Stage 3 culture media at a concentration of 2-20 ng/ml, 5-15 ng/ml, or 7.5-12.5 ng/ml. In one embodiment, the IGF-1 pathway agonist is IGF-1, which is present in the Stage 3 culture media at a concentration of 10 ng/ml.

Polyunsaturated fatty acids (PFA) are fat molecules that have more than one unsaturated carbon bond in the molecule. In some embodiments, the PFA is a methylene-interrupted polyene. In some embodiments, the methylene-interrupted polyene is linoleic acid, α-linoleic acid (ALA), stearidonic acid (SDA), eicosapentaenoic acid (EPA), docosapentaenoic acid (DPA), docosahexaenoic acid (DHA), γ-linoleic acid (GLA), dihomo-γ-linoleic acid (DGLA), hexadecatrienoic acid (HTA), eicosatrienoic acid (ETE), eicosatetraenoic acid (ETA), heneicosapentaenoic acid (HPA), tetracosapentaenoic acid, tetracosahexaenoic acid, eicosadienoicd acid, arachidonic acid (AA), docosadienoic acid, adrenic acid (AdA), tetracosatetraenoic acid, or tetracosapentaenoic acid. In some embodiments, the PFA is a conjugated fatty acid. In some embodiments, the conjugated fatty acid is conjugated linoleic acid (CLA), conjugated linolenic acid, rumelenic acid, α-parinaric acid, β-parinaric acid, or bosseopentaenoic acid. In some embodiments, the PFA is pinolenic acid or sciadonic acid.

In some embodiments, the PFA is selected from the group consisting of linoleic acid, cx-linoleic acid (ALA), stearidonic acid (SDA), cicosapentaenoic acid (EPA), docosapentaenoic acid (DPA), docosahexaenoic acid (DHA), γ-linoleic acid (GLA), dihomo-γ-linoleic acid (DGLA), hexadecatrienoic acid (HTA), eicosatrienoic acid (ETE), eicosatetraenoic acid (ETA), heneicosapentaenoic acid (HPA), tetracosapentaenoic acid, tetracosahexaenoic acid, eicosadienoicd acid, arachidonic acid (AA), docosadienoic acid, adrenic acid (AdA), tetracosatetraenoic acid, tetracosapentaenoic acid, conjugated linoleic acid (CLA), conjugated linolenic acid, rumelenic acid, α-parinaric acid, β-parinaric acid, bosseopentaenoic acid, pinolenic acid, sciadonic acid, and combinations thereof. In one embodiment, the PFA is present in the culture media at a concentration within a range of 25-200 μM, 50-150 μM, or 75-125 μM, or at a concentration of 100 μM. In one embodiment, the PFA is linoleic acid. In one embodiment, the PFA is linoleic acid, which is present in the Stage 3 culture media at a concentration of within a range of 25-200 μM, 50-150 μM, or 75-125 μM. In one embodiment, the PFA is linoleic acid, which is present in the Stage 3 culture media at a concentration of 100 μM.

Agonists of the TrkC (tropomyosin-related kinase receptor C) pathway include agents, molecules, compounds, or substances capable of stimulating (activating) signaling through the TrkC pathway. In some embodiments, a TrkC pathway agonist binds to a TrkC receptor and stimulates or increases the activity of or through the TrkC receptor. In some examples, a TrkC pathway agonist is capable of (i) increasing phosphorylation of AKT or (ii) increasing phosphorylation of ERK. In some embodiments, the TrkC pathway agonist is selected from the group consisting of neurotrophin-3 (NT-3), peptidomimetics based on β-turns of NT-3, LM22B 10, GNF 5837, and combinations thereof. In one embodiment, the TrkC pathway agonist is NT-3. In one embodiment, the TrkC pathway agonist is present in the culture media at a concentration within a range of 2-20 ng/ml, 5-15 ng/ml, or 7.5-12.5 ng/ml. In one embodiment, the TrkC pathway agonist is NT-3, which is present in the Stage 4 culture media at a concentration of 2-20 ng/ml, 5-15 ng/ml, or 7.5-12.5 ng/ml. In one embodiment, the TrkC pathway agonist is NT-3, which is present in the Stage 4 culture media at a concentration of 10 ng/ml.

Agonists of thyroid hormone receptor (THR) include agents, molecules, compounds, or substances capable of stimulating (activating) signaling through the thyroid hormone receptor pathway. In some embodiments, a THR pathway agonist binds to a THR (e.g., THRα or THRβ) and stimulates or increases the activity of or through the THR. In some examples, a THR pathway agonist is capable of (i) increasing phosphorylation of AKT or (ii) increasing phosphorylation of mTOR. In some embodiments, the THR pathway agonist is a THRα-selective agonist. In some embodiments, the THRα-selective agonist is CO23. In some embodiments, the THR pathway agonist is a THRβ-selective agonist. In some embodiments, the THRβ-selective agonist is Resmetirom, Sobetirome, Sob-AM2, ZTA-261, MB-07811, MB-07344, or ALG-055009. In some embodiments, the THR pathway agonist binds to both THRα and THRβ. In some embodiments, the agent that binds to both THRα and THRβ is T3, T4, Tiratricol, Liothyronine, Octinoxate, 3,5-Diiodothyropropinonic acid, or Eprotirome.

In some embodiments, the THR pathway agonist is selected from the group consisting of T3, T4, Tiratricol, Liothyronine, Octinoxate, 3,5-Diiodothyropropinonic acid, Eprotirome, CO23, Resmetirom, Sobetirome, Sob-AM2, ZTA-261, MB-07811, MB-07344, ALG-055009, and combinations thereof. In one embodiment, the THR agonist is present in the culture media at a concentration within a range of 50-150 nM, 75-125 nM, 90-110 nM, or at 100 nM. In one embodiment, the THR agonist is T3. In one embodiment, the THR agonist is T3, which is present in the Stage 4 culture media at a concentration of 50-150 nM, 75-125 nM, 90-110 nM, or at 100 nM. In one embodiment, the THR agonist is T3, which is present in the Stage 4 culture media at a concentration of 100 nM.

PKA pathway agonists include agents, molecules, compounds, or substances capable of stimulating (activating) signaling through the PKA pathway. In some embodiments, a PKA pathway agonist binds to a PKA and stimulates or increases the activity of or through the PKA. In some examples, a PKA pathway agonist is capable of (i) increasing phosphorylation of CREB or (ii) increasing expression of c-Fos. In some embodiments, the PKA pathway agonist is selected from the group consisting of cAMP, Dibutyryl-cAMP, 8-Br-CAMP, CAMPS-Sp, CW 008, Forskolin, 8-CPT-CAMP, Adenosine 3′,5′-cyclic Monophosphate, N6-Benzoyl-cAMP, Sodium Salt, Adenosine 3′, 5′-cyclic monophosphate sodium salt monohydrate, (S)-Adenosine, cyclic 3′, 5′-(hydrogenphosphorothioate)triethylammonium, Sp-Adenosine 3′, 5′-cyclic monophosphorothioate triethylammonium salt, Sp-5,6-DCI-cBiMPS, 8-Bromoadenosine 3′, 5′-cyclic Monophosphothioate, Sp-Isomer sodium salt, Adenosine 3′, 5′-cyclic Monophosphorothioate, 8-Bromo-cAMP, Sp-Isomer, Sp-8-pCPT-cyclic GMPS Sodium, 8-Bromoadenosine 3′, 5′-cyclic monophosphate, N6-Monobutyryladenosine 3′: 5′-cyclic monophosphate sodium salt, 8-PIP-CAMP, Sp-CAMPS, and combinations thereof. In one embodiment, the PKA pathway agonist is present in the culture media at a concentration within a range of 0.5-2.5 μM, 0.75-2.0 μM, or 1.0-1.5 μM, or at a concentration of 1.0 μM. In one embodiment, the PKA pathway agonist is cAMP. In one embodiment, the PKA pathway agonist is cAMP, which is present in the Stage 4 culture media at a concentration of 0.5-2.5 μM, 0.75-2.0 μM, or 1.0-1.5 μM. In one embodiment, the PKA pathway agonist is cAMP, which is present in the Stage 4 culture media at a concentration of 1.0 μM.

Short chain fatty acids (SCFAs) are fatty acids of two to six carbon atoms. In some embodiments, the SCFA is selected from the group consisting of propionate (propionic acid), acetate (acetic acid), butyrate (butyric acid), valerate (valeric acid), isobutyrate (isobutyric acid), isovalerate (isovaleric acid), 2-methylbutanoate (2-methylbutyric acid), and combinations thereof. In one embodiment, the SCFA is present in the culture media at a concentration within a range of 50-150 nM, 75-125 nM, or 90-110 nM, or at 100 nM. In one embodiment, the SCFA is propionate. In one embodiment, the SCFA is propionate, which is present in the Stage 4 culture media at a concentration of 50-150 nM, 75-125 nM, 90-110 nM, or at 100 nM. In one embodiment, the SCFA is propionate, which is present in the Stage 4 culture media at a concentration of 100 nM.

Additional agents that are used in the Stage 1 and/or Stage 2 culture media for generating OPCs from pluripotent stem cells are described below.

Agonists of the Akt pathway include agents, molecules, compounds, or substances capable of stimulating (activating) the signaling pathway of one or more of the serine/threonine kinase Akt family members, which include Akt1 (also designated PKB or RacPK), Akt2 (also designated PKBβ or RacPK-β), and Akt 3 (also designated PKBγ or thyoma viral proto-oncogene 3). In some examples, an Akt pathway agonist is capable of (i) increasing phosphorylation of PI3K, (ii) increasing phosphorylation of one or more AKT kinases, (iii) increasing phosphorylation of GSK3β, (iv) increasing phosphorylation of S6, (v) increasing phosphorylation of mTOR, or (vi) increasing phosphorylation of S6K1. In some embodiments, the Akt pathway agonist is Sc79, Demethyl-Coclaurine, LM22B-10, YS-49, YS-49 monohydrate, Demethylasterriquinone B1, Recilisib, N-Oleyol glycine, NSC45586 sodium, Periplocin, CHPG sodium salt, Bilobalide, 6-hydorxyflavone, Musk ketone, SEW2871, 8-Prenylnaringenin, or Razuprotafib. In one embodiment, the Akt pathway agonist is present in the culture media at a concentration within a range of 0.1-10 μM, 0.5-5 μM, 0.5-2.5 μM, or 0.5-1.5 μM or at a concentration of 1 μM or 2 μM. In one embodiment, the Akt pathway agonist is SC79. In one embodiment, the Akt pathway agonist is SC79, which is present in the culture media at a concentration of 0.1-10 μM, 0.5-5 μM, 0.5-2.5 μM, or 0.5-1.5 μM. In one embodiment, the Akt pathway agonist is SC79, which is present in the culture media at a concentration of 1 μM (Stage 1 media) or 2 μM (Stage 2 media).

Agonists of the mTOR (mammalian target of rapamycin) pathway include agents, molecules, compounds, or substances capable of stimulating (activating) signaling through mTOR, a PI3K-related kinase family member which is a core component of the mTORC1 and mTORC2 complexes. In some examples, an mTOR pathway agonist is capable of (i) increasing expression of mTOR, (ii) increasing phosphorylation of mTOR, (iii) increasing phosphorylation of S6 kinase 1 (S6K1), (iv) increasing phosphorylation of eukaryotic initiation factor 4E-binding protein 1 (4EBP1), (v) increasing phosphorylation of AKT, (vi) increasing phosphorylation of insulin-like growth factor I receptor (IGF-IR), or (vii) increasing phosphorylation of insulin receptor. In some embodiments, the mTOR pathway agonist is a leucine or its analog. In some embodiments, the leucine analog is L-Leucine, NV-5138, NV-5138 Hydrochloride, L-Leucine-d₁, L-Leucine-2-¹³C,¹⁵N, Leucine-¹³C₆, L-Leucine-d₇, L-Leucine-d₁₀, L-Leucine-d₂, L-Leucine-d₃, L-Leucine-¹⁸O₂, L-Leucine-¹³C, L-Leucine-2-¹³C, L-Leucine-¹³C₆-¹⁵N, L-Leucine-¹⁵N, or L-Leucine-1-¹³C,¹⁵N. In some embodiments, the mTOR pathway agonist activates the AKT/mTOR pathway. In some embodiments, the AKT/mTOR pathway activator is 3-benzyl-5-((2-nitrophenoxy)methyl)-dihydrofuran-2 (3H)-one (3BDO), Salidroside, or Testosterone. In some embodiments, the mTOR pathway agonist binds an mTORC1 and/or an mTORC2 and stimulates or increases signaling through or activity of the mTORC1 and/or the mTORC2. In some embodiments, the mTOR pathway agonist binds the ATP domain of mTORC. In some embodiments, the ATP domain-binding agent is MHY1485.

In some embodiments, the mTOR pathway agonist is selected from the group consisting of MHY1485, 3BDO, Salidroside, testosterone, L-Leucine, NV-5138, NV-5138 Hydrochloride, L-Leucine-d₁, L-Leucine-2-¹³C,¹⁵N, Leucine-¹³C₆, L-Leucine-d₇, L-Leucine-d₁₀, L-Leucine-d₂, L-Leucine-d₃, L-Leucine-¹⁸O₂, L-Leucine-¹³C, L-Leucine-2-¹³C, L-Leucine-¹³C₆-¹⁵N, L-Leucine-¹⁵N, or L-Leucine-1-¹³C,¹⁵N, and combinations thereof. In one embodiment, the mTOR pathway agonist is present in the culture media at a concentration within a range of 0.1-10 μM, 0.5-5 μM, 0.5-2.5 μM, or 0.5-1.5 μM or at a concentration of 1 μM. In one embodiment, the mTOR pathway agonist is MHY1485. In one embodiment, the mTOR pathway agonist is MHY1485, which is present in the culture media at a concentration of 0.1-10 μM, 0.5-5 μM, 0.5-2.5 μM, or 0.5-1.5 μM. In one embodiment, the mTOR pathway agonist is MHY1485, which is present in the culture media at a concentration of 1 μM.

Antagonists of the WNT pathway include agents, molecules, compounds, or substances capable of inhibiting (downregulating) the canonical Wnt/β-catenin signaling pathway, which biologically is activated by binding of a Wnt-protein ligand to a Frizzled family receptor. In some examples, a WNT pathway antagonist is capable of (i) reducing expression of WNT, (ii) reducing expression of FZD, (iii) reducing phosphorylation of low-density lipoprotein-related receptors 5 and 6 (LRP5/6), (iv) reducing phosphorylation of dishevelled (DVL), or (v) increasing phosphorylation of β-catenin. In some embodiments, the WNT pathway antagonist prevents secretion of WNT ligands by inhibiting Porcupine (PORCN). In some embodiments, the PORCN inhibitor is IWP-2, IWP-4, C59, WNT974, CGX1321, ETC-159, RXC004, or GNF-6231. In some embodiments, the WNT pathway antagonist is an agent targeting the ligands of FZD receptors. In some embodiments, the ligand-targeting agent is WIF-1 or Ipafricept. In some embodiments, the WNT pathway antagonist inhibits LRP5/6. In some embodiments, the LRP5/6 inhibitor is DKK1, BMD4503-2, or Salinomycin. In some embodiments, the WNT pathway antagonist inhibits DVL. In some embodiments, the DVL inhibitor is NSC668036, FJ9, or 3289-8625. In some embodiments, the WNT pathway antagonist inhibits tankyrases. In some embodiments, the tankyrase inhibitor is XAV939, IWR-1-endo, JW55, JW74, WIKI4, TC-E 5001, NVP-TNKS656, or LZZ-02. In some embodiments, the WNT pathway antagonist destabilizes the β-catenin/T cell factor (TCF) complex. In some embodiments, the β-catenin/TCF complex-targeting agent is MSAB, CCT251545, KY02111, LF3, FH535, KYA1797K, iCRT3, PNU-74654, Cardionogen 1, CCT036477, KY1220, Triptonide, iCRT5, iCRT14, or PKF118-310. In some embodiments, the WNT pathway antagonist inhibits CREB binding protein (CBP). In some embodiments, the CBP inhibitor is ICG001, JW67, NLS-StAx-h, PRI-724, or GNE-781. In some embodiments, the WNT pathway antagonist inhibits MET receptor signaling. In some embodiments, the MET inhibitor is Capmatinib. In some embodiments, the WNT pathway antagonist inhibits TRAF2 and NCK interacting kinase (TNIK). In some embodiments, the TNIK inhibitor is NCB-0846. In some embodiments, the WNT pathway antagonist inhibits casein kinase I (CK1). In some embodiments, the CK1 inhibitor is TAK715. In some embodiments, the WNT pathway antagonist inhibits pyruvate flavodoxin/ferredoxin oxidoreductases (PFORs). In some embodiments, the PFOR inhibitor is Nitazoxanide. In some embodiments, the WNT pathway antagonist binds a FZD and inhibits or decreases signaling through or activity of the FZD. In some embodiments, the WNT antagonist is an antibody targeting FZDs. In some embodiments, the FZD-targeting antibody is Vantictumab. In some embodiments, the WNT pathway antagonist is an antagonist of FZDs. In some embodiments, the FZD antagonist is OTSA-101 or Fz7-21.

In some embodiments, the WNT pathway antagonist is selected from the group consisting of XAV939, IWR-1-endo, JW55, JW74, WIKI4, TC-E 5001, NVP-TNKS656, LZZ-02, ICG001, JW67, NLS-StAx-h, PRI-724, GNE-781, Capmatinib, IWP-2, IWP-4, C59, WNT974, CGX1321, ETC-159, RXC004, GNF-6231, MSAB, CCT251545, KY02111, LF3, FH535, KYA1797K, iCRT3, PNU-74654, Cardionogen 1, CCT036477, KY1220, Triptonide, iCRT5, iCRT14, PKF118-310, NCB-0846, Triptonide, TAK715, WIF-1, Ipafricept, DKK1, BMD4503-2, Salinomycin, NSC668036, FJ9, 3289-8625, Nitazoxanide, Vantictumab, OTSA-101, F27-21, and combinations thereof. In one embodiment, the WNT pathway antagonist is present in the culture media at a concentration within a range of 10-500 nM, 50-250 nM, or 50-150 nM or at a concentration of 100 nM. In one embodiment, the WNT pathway antagonist is XAV939. In one embodiment, the WNT pathway antagonist is XAV939, which is present in the culture media at a concentration of 10-500 nM, 50-250 nM, or 50-150 nM. In one embodiment, the WNT pathway antagonist is XAV939, which is present in the culture media at a concentration of 100 nM.

Agonists of the SHH (sonic hedgehog) pathway include agents, molecules, compounds, or substances capable of stimulating (activating) signaling through the SHH pathway, which biologically involves binding of SHH to the Patched-1 (PTCH1) receptor and transduction through the Smoothened (SMO) transmembrane protein. In some examples, an SHH pathway agonist is capable of (i) increasing expression of SHH, (ii) increasing cell surface expression of SMO receptors, or (iii) increasing expression of Gli1. In some embodiments, an SHH pathway agonist binds a PTCH1 receptor or SMO receptor and stimulates or increases signaling through or activity of the PTCH1 receptor or SMO receptor. In some embodiments, the SHH pathway agonist is an agonist of PTCH1 receptor. In some embodiments, the PTCH1 receptor agonist is SHH. In some embodiments, the SHH pathway agonist is an agonist of SMO receptor. In some embodiments, the SMO receptor agonist is Purmorphamine, GSA 10, or SAG.

In some embodiments, the SHH pathway agonist is selected from the group consisting of Purmorphamine, GSA 10, SAG, SHH, and combinations thereof. In one embodiment, the SHH pathway agonist is present in the culture media at a concentration within a range of 100-1000 nM, 250-750 nM, or 400-600 nM or at a concentration of 500 nM. In one embodiment, the SHH pathway antagonist is Purmorphamine. In one embodiment, the SHH pathway antagonist is Purmorphamine, which is present in the culture media at a concentration of 100-1000 nM, 250-750 nM, or 400-600 nM. In one embodiment, the SHH pathway antagonist is Purmorphamine, which is present in the culture media at a concentration of 500 nM.

Antagonists of the BMP (bone morphogenetic protein) pathway include agents, molecules, compounds, or substances capable of inhibiting (downregulating) the BMP signaling pathway, which biologically is activated by binding of BMP to a BMP receptor, which are activin receptor-like kinases (ALK) (e.g., type I BMP receptor, including but not limited to ALK2 and ALK3). In some examples, a BMP pathway antagonist is capable of (i) reducing phosphorylation of Smad 1/5/8 induced by BMP, (ii) reducing expression of Smad1/5/8, (iii) reducing phosphorylation of ERK induced by BMP, or (iv) reducing expression of Id1. In some embodiments, the BMP pathway antagonist binds BMPs. In some embodiments, the BMP-binding agent is follistatin, Noggin, follistatin-like 1, Chordin, Ventroptin, Twisted gastrulation, Dan, Cerberus, PRDC, Dante, Caronte, Gremlin, or Sclerostin. In some embodiments, the BMP pathway antagonist binds a BMP receptor and inhibits or decreases signaling through or activity of the BMP receptor. In some embodiments, the BMP pathway antagonist is a selective BMP type I receptor inhibitor. In some embodiments, the BMP type I receptor inhibitor is LDN193189, DMH1, DMH2, Dorsomorphin, K02288, LDN214117, LDN212854, or ML 347.

In some embodiments, the BMP pathway antagonist is selected from the group consisting of LDN193189, DMH1, DMH2, Dorsopmorphin, K02288, LDN214117, LDN212854, ML347, follistatin, Noggin, follistatin-like 1, Chordin, Ventroptin, Twisted gastrulation, Dan, Cerberus, PRDC, Dante, Caronte, Gremlin, Sclerostin, and combinations thereof. In one embodiment, the BMP pathway antagonist is present in the culture media at a concentration within a range of 100-1000 nM, 150-750 nM, 100-500 nM, or 150-350 nM or at a concentration of 250 nM. In one embodiment, the BMP pathway antagonist is LDN193189. In one embodiment, the BMP pathway antagonist is LDN193189, which is present in the culture media at a concentration of 100-1000 nM, 150-750 nM, 100-500 nM, or 150-350 nM. In one embodiment, the BMP pathway antagonist is LDN193189, which is present in the culture media at a concentration of 250 nM.

Antagonists of the PKC (protein kinase C) pathway include agents, molecules, compounds, or substances capable of inhibiting (downregulating) a PKC signaling pathway, which biologically is mediated by a PKC family member. The PKC family of serine/threonine kinases comprises 15 isozymes, including the “classical” PKC subcategory, which contain the isoforms α, β1, β2, and γ. In one embodiment, the PKC pathway antagonist inhibits the activity of at least one (and in other embodiments, at least two or three) PKC enzyme selected from PKCα, PKCβ1, PKCβ2, and PKCγ. In some examples, a PKC pathway antagonist is capable of (i) reducing phosphorylation of PKC, (ii) reducing expression of PKC, (iii) inhibiting phosphorylation of MARCKS, or (iv) inhibiting phosphorylation of S6. In some embodiments, the PKC pathway antagonist is a non-competitive inhibitor. In some embodiments, the non-competitive PKC inhibitor is [Ala¹¹³]-MBP, [Ala¹⁰⁷]-MBP, or Piccatannol. In some embodiments, the PKC pathway antagonist binds one or more PKC isozymes and inhibits or decreases signaling through or activity of the one or more PKC isozymes. In some embodiments, the PKC pathway antagonist is a pan-inhibitor. In some embodiments, the pan PKC inhibitor is Go 6983, PKC 412 (Midostaurin), Bis II, Go 6976, K-252c, D-erythro-Sphingosine, Chelerythrine chloride (NSC 646662), Bis I (GF 109203X), Calphostin C, TCS 21311, Bis XI hydrochloride (Ro 32-0432), Enzastaurin (LY317615), Sotrastaurin (AEB071), Hypocrellin A, HA-100, HA-100 dihydrochloride, Hypericin, AM-2282, Daphnetin, Dequalinium Chloride, Rottlerin, Darovasertib, Bis IV, Bis VIII, Bis IX, Bis X, Bis X hydrochloride, Bis XI, K-252a, K-252b, K-252d, Chelerythrine, Mitoxantrone, Verbascoside, CGP60474, Ro 31-8220 mesylate, Valrubicin, KT5823, Psychosine, SB-218078, H-7, Sphingosine, A-3 hydrochloride, TAS-301, SB 220025, Fasudil, PKC-IN-4, N-Desmethyltamoxifen, N-Desmethyltamoxifen hydrochloride, PKC-IN-1, PF-04577806, PF-03622905, Balanol, HBDDE, Afuresertib, NPC-15437 dihydrochloride, Safingol, Resveratrol, Riluzole, UCN-01, 7-Oxostaurosporine, Melittin, Hispidin, PKC19-31, PKC19-36, Go 7874, or Ilmofosine. In some embodiments, the PKC pathway antagonist is specific to conventional PKC isozymes. In some embodiments, the PKCα, PKCβ, and/or PKCγ-specific PKC inhibitor is PKC β pseudosubstrate, CGP-53353, LY333531, CMPD101, Bis III, Evo312, Leucosceptoside A, ML192, C2-1, C2-2, or C2-4. In some embodiments, the PKC pathway antagonist is specific to novel PKC isozymes. In some embodiments, the PKCδ, PKCε, PKCη, and/or PKCθ-specific PKC inhibitor is VTX-27, PKC ε pseudosubstrate, Epsilon-V1-2, CC-90005, Delcasertib, KAI-1678, CDK8-IN-12, CIDD-0072424, BJE6-106, or AS2521780. In some embodiments, the PKC pathway antagonist is specific to atypical PKC isozymes. In some embodiments, the PKCζ, PKCι, and/or PKCλ-specific PKC inhibitor is CRT0066854, CRT0066854 hydrochloride, auranofin, aurothiomalate sodium, ICA-1, ζ-Stat, ZIP, ACPD, DNDA, or PS432. In some embodiments, the PKC pathway antagonist is an antisense oligonucleotide. In some embodiments, the antisense oligonucleotide is Aprinocarsen.

In some embodiments, the PKC pathway antagonist is selected from the group consisting of Go 6983, Sotrastaurin, Enzastaurin (LY31615), Staurosporine (AM-2282), Go 6976, BIS I (GF 109203X), Ro 31-8220 Mesylate, PKC 412 (Midostaurin), Bis II, K-252c, D-erythro-Sphingosine, Chelerythrine chloride (NSC 646662), Calphostin C, TCS 21311, Bis XI hydrochloride (Ro 32-0432), Hypocrellin A, HA-100, HA-100 dihydrochloride, Hypericin, Daphnetin, Dequalinium Chloride, Rottlerin, Darovasertib, Bis IV, Bis VIII, Bis IX, Bis X, Bis X hydrochloride, Bis XI, K-252a, K-252b, K-252d, Chelerythrine, Mitoxantrone, Verbascoside, CGP60474, Valrubicin, KT5823, Psychosine, SB-218078, H-7, Sphingosine, A-3 hydrochloride, TAS-301, SB 220025, Fasudil, PKC-IN-4, N-Desmethyltamoxifen, N-Desmethyltamoxifen hydrochloride, PKC-IN-1, PF-04577806, PF-03622905, Balanol, HBDDE, Afuresertib, NPC-15437 dihydrochloride, Safingol, Resveratrol, Riluzole, UCN-01, 7-Oxostaurosporine, Melittin, Hispidin, PKC19-31, PKC19-36, Go 7874, Ilmofosine, PKC β pseudosubstrate, CGP-53353, LY333531, CMPD101, Bis III, Evo312, Leucosceptoside A, ML192, C2-1, C2-2, C2-4, VTX-27, PKC & pseudosubstrate, Epsilon-V1-2, CC-90005, Delcasertib, KAI-1678, CDK8-IN-12, CIDD-0072424, BJE6-106, AS2521780, CRT0066854, CRT0066854 hydrochloride, auranofin, aurothiomalate sodium, ICA-1, ζ-Stat, ZIP, ACPD, DNDA, PS432, Aprinocarsen, [Ala¹¹³]-MBP, [Ala¹⁰⁷]-MBP, Piccatannol, and combinations thereof. In one embodiment, the PKC pathway antagonist is present in the culture media at a concentration within a range of 10-500 nM, 50-300 nM, 50-150 nM, or 75-150 nM or at a concentration of 110 nM. In one embodiment, the PKC pathway antagonist is Go 6983. In one embodiment, the PKC pathway antagonist is Go 6983, which is present in the culture media at a concentration of 10-500 nM, 50-300 nM, 50-150 nM, or 75-150 nM. In one embodiment, the PKC pathway antagonist is Go 6983, which is present in the culture media at a concentration of 110 nM.

Antagonists of the AKT pathway include agents, molecules, compounds, or substances capable of inhibiting (downregulating) the signaling pathway of one or more of the serine/threonine kinase AKT family members, which include AKT1 (also designated PKB or RacPK), AKT2 (also designated PKBβ or RacPK-β), and AKT 3 (also designated PKBγ or thymoma viral proto-oncogene 3). In some examples, an AKT pathway antagonist is capable of (i) reducing phosphorylation of PI3K, (ii) reducing phosphorylation of one or more AKT kinases, (iii) reducing phosphorylation of GSK3β, (iv) reducing phosphorylation of S6, (v) reducing phosphorylation of mTOR, or (vi) reducing phosphorylation of S6K1. In some embodiments, the AKT pathway antagonist is a pan-PI3K inhibitor. In some embodiments, the pan-PI3K inhibitor is Pictilisib, Buparlisib, Pilaralisib, BAY806946, or PX-866. In some embodiments, the AKT pathway antagonist is an isoform-selective PI3K inhibitor. In some embodiments, the isoform-selective PI3K inhibitor is Alpclisib, Tasclisib, IPI-145, SAR-260301, MLN-1117, GSK2636771, Idelsalisib, or AMG319. In some embodiments, the AKT pathway antagonist is a dual pan-PI3K and mTOR inhibitor. In some embodiments, the dual pan-PI3K and mTOR inhibitor is PF-04691502, SF1126, Dactolisib, Voxtalisib, GSK1059615, GDC-0980, GSK2126458, PF-05212384, or BGT-226. In some embodiments, the AKT pathway antagonist is an mTORC1/mTORC2 inhibitor. In some embodiments, the mTORC1/mTORC2 inhibitor is Rapamycin, Temsirolimus, Everolimus, Deforolimus, AZD8055, AZD2014, MLN-128, or CC-223. In some embodiments, the AKT pathway antagonist binds one or more AKT kinases and inhibits or reduces signaling through or activity of the one or more AKT kinases. In some embodiments, the AKT pathway antagonist is MK2206, GSK690693, Perifosine (KRX-0401), Ipatasertib (GDC-0068), Capivasertib (AZD5363), AKTi-1/2, AT 7867, CCT128930, A-674563, PHT-427, Miltefosine, AT 13148, ML-9, BAY1125976, Oridonin, TIC10, Pectolinarin, Miransertib, AKTI IV, 10-DEBC, API-1, SC66, FPA-124, Triciribine (API-2), Urolithin A, ARQ751, Borussertib, Uprosertibe, Afuresertib, VQD-002, M2698, SR13668, ZSTK474, or Cenisertib.

In some embodiments, the AKT pathway antagonist is selected from the group consisting of MK2206, GSK690693, Perifosine (KRX-0401), Ipatasertib (GDC-0068), Capivasertib (AZD5363), AKTi-1/2, AT 7867, CCT128930, A-674563, PHT-427, Miltefosine, AT 13148, ML-9, BAY1125976, Oridonin, TIC10, Pectolinarin, Miransertib, AKTI IV, 10-DEBC, API-1, SC66, FPA-124, Triciribine (API-2), Urolithin A, ARQ751, Borussertib, Uprosertibe, Afuresertib, VQD-002, M2698, SR13668, ZSTK474, Cenisertib, PF-04691502, SF1126, Dactolisib, Voxtalisib, GSK1059615, GDC-0980, GSK2126458, PF-05212384, BGT-226, Rapamycin, Temsirolimus, Everolimus, Deforolimus, AZD8055, AZD2014, MLN-128, CC-223, Pictilisib, Buparlisib, Pilaralisib, BAY806946, PX-866, Alpelisib, Tasclisib, IPI-145, SAR-260301, MLN-1117, GSK2636771, Idelsalisib, AMG319, and combinations thereof. In one embodiment, the AKT pathway antagonist is present in the culture media at a concentration within a range of 25-300 nM, 50-250 nM, 75-200 nM, or 100-150 nM or at a concentration of 125 nM. In one embodiment, the AKT pathway antagonist is MK2206. In one embodiment, the AKT pathway antagonist is MK2206, which is present in the culture media at a concentration within a range of 25-300 nM, 50-250 nM, 75-200 nM, or 100-150 nM. In one embodiment, the AKT pathway antagonist is MK2206, which is present in the culture media at a concentration of 125 nM.

Antagonists of the mTOR (mammalian target of rapamycin) pathway include agents, molecules, compounds, or substances capable of inhibiting (downregulating) an mTOR signaling pathway, wherein mTOR is a PI3K-related kinase family member which is a core component of the mTORC1 and mTORC2 complexes. In some examples, an mTOR pathway antagonist is capable of (i) reducing expression of mTOR, (ii) reducing phosphorylation of mTOR, (iii) reducing phosphorylation of S6 kinase 1 (S6K1), (iv) reducing phosphorylation of eukaryotic initiation factor 4E-binding protein 1 (4EBP1), (v) reducing phosphorylation of AKT, (vi) reducing phosphorylation of insulin-like growth factor 1 receptor (IGF-1R), or (vii) reducing phosphorylation of insulin receptor. In some embodiments, the mTOR pathway antagonist suppresses the formation of mTOR complexes. In some embodiments, the agent that suppresses mTORC1 and/or mTORC2 formation is BC-LI-0186 or Dihydromyricetin. In some embodiments, the mTOR pathway antagonist reduces the protein expression of mTOR. In some embodiments, the agent that reduces protein level of mTOR is Niclosamide or Zederone. In some embodiments, the mTOR pathway antagonist reduces the phosphorylation of mTOR. In some embodiments, the agent that reduces phosphorylation of mTOR is TML-6. In some embodiments, the mTOR pathway antagonist binds an mTORC1 and/or an mTORC2 and inhibits or decreases signaling through or activity of the mTORC1 and/or the mTORC2. In some embodiments, the mTOR pathway antagonist inhibits both mTORC1 and mTORC2. In some embodiments, the mTORC1/mTORC2 dual inhibitor is AZD3147, AZD8055, eCF309, Torin 1, Torin 2, WYE-687, XL388, STK16-IN-1, Torkinib (PP 242), Sapanisertib, Vistusertib, KU-0063794, WYE-132, Onatasertib, OSI-027, CZ415, AZD2014, WYE-354, WAY-600, Palomid 529, GDC-0349, PQR620, MTI-31, FT-1518, CC214-2, or Pomiferin. In some embodiments, the mTOR pathway antagonist selectively inhibits mTORC1. In some embodiments, the mTORC1-selective inhibitor is Rapamycin, Everolimus, Temsirolimus, Ridaforolimus, Umirolimus, Zotarolimus, RMC-5552, RapaLink-1, MT 63-78, or Coronarin A. In some embodiments, the mTOR pathway antagonist selectively inhibits mTORC2. In some embodiments, the mTORC2-selective inhibitor is JR-AB2-011 or Aloe emodin. In some embodiments, the mTOR pathway antagonist inhibits both mTOR and phosphoinositide 3-kinases (PI3Ks). In some embodiments, the mTOR/PI3K dual inhibitor is Dactolisib, PI-103, Omipalisib, PF 04691502, Gedatolisib (PF 05212384), Samotolisib, Voxtalisib, Paxalisib, Apitolisib, BGT226, GSK1059615, Bimiralisib, PP121, PKI-402, VS-5584, GNE-317, PQR530, GNE-493, PKI-179, NSC781406, PF 04979064, GNE-477, FD274, GNE-490, or SN32976. In some embodiments, the mTOR pathway antagonist inhibits both mTOR and DNA-dependent protein kinase (DNA-PK). In some embodiments, the DNA-PK/mTOR dual inhibitor is CC-115. In some embodiments, the mTOR pathway antagonist inhibits mTOR, PI3K, and DNA-PK. In some embodiments, the PI3K/DNA-PK/mTOR triple inhibitor is KU-57788 (NU7441) or ETP-45658. In some embodiments, the mTOR pathway antagonist inhibits both mTOR and ataxia telangiectasia and Rad3 related (ATR) protein. In some embodiments, the mTOR/ATR dual inhibitor is ETP-46464.

In some embodiments, the mTOR pathway antagonist is selected from the group consisting of AZD3147, AZD8055, eCF309, Torin 1, Torin 2, WYE-687, XL388, STK16-IN-1, Torkinib (PP 242), Sapanisertib, Vistusertib, KU-0063794, WYE-132, Onatasertib, OSI-027, CZ415, AZD2014, WYE-354, WAY-600, Palomid 529, GDC-0349, PQR620, MTI-31, FT-1518, CC214-2, Pomiferin, Dactolisib, PI-103, Omipalisib, PF 04691502, Gedatolisib (PF 05212384), Samotolisib, Voxtalisib, Paxalisib, Apitolisib, BGT226, GSK1059615, Bimiralisib, PP121, PKI-402, VS-5584, GNE-317, PQR530, GNE-493, PKI-179, NSC781406, PF 04979064, GNE-477, FD274, GNE-490, SN32976, Rapamycin, Everolimus, Temsirolimus, Ridaforolimus, Umirolimus, Zotarolimus, RMC-5552, RapaLink-1, MT 63-78, Coronarin A, NU7441, ETP-45658, BC-LI-0186, Dihydromyricetin, Niclosamide, Zederone, TML-6, JR-AB2-011, Aloe emodin, CC-115, ETP-46464, and combinations thereof. In one embodiment, the mTOR pathway antagonist is present in the culture media at a concentration within a range of 5-25 nM, 10-20 nM, or 12.5-17.5 nM or at a concentration of 15 nM. In one embodiment, the mTOR pathway antagonist is AZD3147. In one embodiment, the mTOR pathway antagonist is AZD3147, which is present in the culture media at a concentration within a range of 5-25 nM, 10-20 nM, or 12.5-17.5 nM. In one embodiment, the mTOR pathway antagonist is AZD3147, which is present in the culture media at a concentration of 15 nM.

When an agonist or antagonist is used in more than one culture media or step of the method, in one embodiment it is the same agonist or antagonist that is used for each culture media or step in which the agent is present. In another embodiment, different agonists or antagonists that affect the same signaling pathway are used in different culture media or steps of the method. For example, for the NOTCH pathway antagonist in first culture media and second culture media of the method of generating VLMCs from OPCs, in one embodiment the same NOTCH pathway antagonist is used in the first and second culture media. In another embodiment, different NOTCH pathway antagonists are used in the first and second culture media. Similarly, for the RA pathway agonist used in steps (a) and (c) (Stages 1 and 3) of the method of generating VLMCs from PSCs, in one embodiment the same RA pathway agonist is used in steps (a) and (c). In another embodiment, different RA pathway agonists are used in steps (a) and (c). Similarly, for the AKT pathway agonist used in steps (a) and (b) (Stages 1 and 2), in one embodiment the same AKT pathway agonist is used in steps (a) and (b). In another embodiment, different AKT pathway agonists are used in steps (a) and (b). Similarly, for the SHH pathway agonist used in steps (a) and (b) (Stages 1 and 2), in one embodiment the same SHH pathway agonist is used in steps (a) and (b). In another embodiment, different SHH pathway agonists are used in steps (a) and (b). Similarly, for the FGFR pathway agonist used in steps (b) and (c) (Stages 2 and 3), in one embodiment the same FGFR pathway agonist is used in steps (b) and (c). In another embodiment, different FGFR pathway agonists are used in steps (b) and (c). Similarly, for the NOTCH pathway antagonist used in steps (c) and (d) (Stages 3 and 4), in one embodiment the same NOTCH pathway antagonist is used in steps (c) and (d). In another embodiment, different NOTCH pathway antagonists are used in steps (c) and (d).

When an agonist or antagonist is used in more than one culture media or step of the method, in one embodiment it is the same concentration of the same agonist or antagonist that is used for each culture media or step in which the agent is present. In another embodiment, different concentrations of the same agonist or antagonist are used in different culture media steps of the method. For example, for the NOTCH pathway antagonist in first culture media and second culture media of the method of generating VLMCs from OPCs, in one embodiment the same concentration of the same NOTCH pathway antagonist is used in the first and second culture media. In another embodiment, different concentrations of the same NOTCH pathway antagonist are used in the first and second culture media. Similarly, for the RA pathway agonist used in steps (a) and (c) (Stages 1 and 3) of the method of generating VLMCs from PSCs, in one embodiment the same concentration of the same RA pathway agonist is used in steps (a) and (c). In another embodiment, different concentrations of the same RA pathway agonist are used in steps (a) and (c). Similarly, for the AKT pathway agonist used in steps (a) and (b) (Stages 1 and 2), in one embodiment the same concentration of the same AKT pathway agonist is used in steps (a) and (b). In another embodiment, different concentrations of the same AKT pathway agonist are used in steps (a) and (b). Similarly, for the SHH pathway agonist used in steps (a) and (b) (Stages 1 and 2), in one embodiment the same concentration of the same SHH pathway agonist is used in steps (a) and (b). In another embodiment, different concentrations of the same SHH pathway agonist are used in steps (a) and (b). Similarly, for the FGFR pathway agonist used in steps (b) and (c) (Stages 2 and 3), in one embodiment the same concentration of the same FGFR pathway agonist is used in steps (b) and (c). In another embodiment, different concentrations of the same FGFR pathway agonist are used in steps (b) and (c). Similarly, for the NOTCH pathway antagonist used in steps (c) and (d) (Stages 3 and 4), in one embodiment the same concentration of the same NOTCH pathway antagonist is used in steps (c) and (d). In another embodiment, different concentrations of the same NOTCH pathway antagonist are used in steps (c) and (d).

### III. Culture Conditions

In combination with the chemically-defined and optimized culture media described in subsection II above, the methods of generating GPCs and VLMCs of the disclosure utilize standard culture conditions established in the art for cell culture. For example, cells can be cultured at 37° C. and 5% CO2 conditions. Cells can be cultured in standard culture vessels or plates, such as 96-well plates. In certain embodiments, the starting pluripotent stem cells or OPCs are adhered to plates, preferably coated with an extracellular matrix material such as vitronectin. In one embodiment, the stem cells are cultured on a vitronectin coated culture surface (e.g., vitronectin coated 96-well plates). In other embodiments, cells are cultured in suspension.

Pluripotent stem cells can be cultured in commercially-available media prior to differentiation. For example, stem cells can be cultured for at least one day in Essential 8 Flex media (Thermo Fisher #A2858501) prior to the start of the differentiation protocol. In a non-limiting exemplary embodiment, stem cells are passaged onto vitronectin (Thermo Fisher #A14700) coated 96-well plates at 150,000 cells/cm2 density and cultured for one day in Essential 8 Flex media prior to differentiation.

To begin the differentiation protocol, the media the starting cells are being cultured in is changed to a basal differentiation media that has been supplemented with signaling pathway agonists and/or antagonists as described above in subsection II. A basal differentiation media can include, for example, a commercially-available base supplemented with additional standard culture media components needed to maintain cell viability and growth, but lacking serum (the basal differentiation media is a serum-free media) or other exogenously-added growth factors. In a non-limiting exemplary embodiment, a basal differentiation media contains 1×IMDM (Thermo Fisher #12440046), 1×F12 (Thermo Fisher #11765047), poly(vinyl alcohol) (Sigma #p8136) at 1 mg/ml, chemically defined lipid concentrate (Thermo Fisher #11905031) at 1%, 1-thioglycerol (Sigma #M6145) at 450 μM, Insulin (Sigma #11376497001) at 0.7 ug/ml, and transferrin (Sigma #10652202001) at 15 μg/ml. In one embodiment, this basal media is supplemented with Albumax II.

The culture media typically is changed regularly to fresh media. For example, in one embodiment, media is changed every 24 hours.

To generate GPCs or VLMCs, the starting cells are cultured in the optimized culture media for sufficient time for cellular differentiation and expression of GPC or VLMC-associated markers. As described in Example 1, it has been discovered that culture of OPCs in the optimized Stage 3 culture media for six days was sufficient for differentiation of OPCs to multipotential GPCs. Accordingly, in one embodiment, cells are cultured for at least 144 hours in Stage 3 culture media. In other embodiments, the cells are cultured for at least 132, 136, 140, 144, 150, 154, or 158 hours in Stage 3 culture media.

Also as described in Example 1, it has been discovered that further culture of GPCs in the optimized Stage 4 culture media for 28 days was sufficient for cell differentiation to VLMCs. Accordingly, in one embodiment, GPCs are cultured for at least 25 days, 26 days, 27 days, 28 days, 29 days, or 30 days in Stage 4 culture media.

### IV. Uses

The methods and compositions of the disclosure for generating GPCs and VLMCs allow for efficient and robust availability of these cell populations for a variety of uses. For example, the methods and compositions can be used in the study of VLMC development and biology, e.g., to assist in the understanding of leptomeningeal diseases and disorders, such as LMD. For example, the GPCs and VLMCs generated using the methods of the disclosure can be further purified according to methods established in the art using agents that bind to surface markers expressed on the cells.

Accordingly, in one embodiment, the disclosure provides a method of isolating GPCs or VLMCs, the method comprising:
contacting GPCs or VLMCs generated by a method of the disclosure with at least one binding agent that binds to a cell surface marker expressed by the GPCs or VLMCs; and
isolating cells that bind to the binding agent to thereby isolate the GPCs or VLMCs.

In one embodiment, the binding agent is an antibody, e.g., a monoclonal antibody (mAb) that binds to the cell surface marker. Cells that bind the antibody can be isolated by methods known in the art, including but not limited to fluorescent activated cell-sorting (FACS) and magnetic activated cell sorting (MACS).

Cells of the VLMC lineage also are contemplated for use in the treatment of various leptomeningeal-related diseases and disorders, through delivery of the cells to a subject having the disease or disorder. Examples of leptomeningeal-related diseases and disorders include, but are not limited to, LMD, and inflammatory brain disorders involving the leptomeninges.

### V. Compositions

In other aspects, the disclosure provides compositions related to the methods of generating GPCs and VLMCs, including culture media and cell cultures, as well as isolated cells and cell populations.

In one aspect, the disclosure provides a culture media for obtaining GPCs comprising an FGFR pathway agonist, an RA pathway agonist, a WNT pathway agonist, a NOTCH pathway antagonist, a PDGFR pathway agonist, and an IGF-1 pathway agonist (and optionally a PFA). Suitable agonists/antagonists and concentration ranges and amounts are described in Subsection II.

In another aspect, the disclosure provides a culture media for obtaining VLMCs comprising a TrkC pathway agonist, a THR pathway agonist, a PKA pathway agonist, and a NOTCH pathway antagonist (and optionally a SCFA). Suitable agonists/antagonists and concentration ranges and amounts are described in Subsection II.

In another aspect, the disclosure provides an isolated cell culture of human GPCs, the culture comprising: human GPCs cultured in a culture media comprising an FGFR pathway agonist, an RA pathway agonist, a WNT pathway agonist, a NOTCH pathway antagonist, a PDGFR agonist, and an IGF-1 pathway agonist (and optionally a PFA).

In another aspect, the disclosure provides an isolated cell culture of human VLMCs, the culture comprising: human VLMCs cultured in a culture media comprising a TrkC pathway agonist, a THR pathway agonist, a PKA pathway agonist, and a NOTCH pathway antagonist (and optionally a short chain fatty acid).

In another aspect, the disclosure provides isolated human VLMCs. In embodiments, the disclosure provides an isolated human VLMC, wherein the cell:
(i) is derived from a PSC (e.g., an ES cell or iPSC, as described herein) or OPC (e.g., OPC as described herein);
(ii) has a fibroblast-like morphology (e.g., bipolar or multipolar, with a flattened, elongated, or spindle-like shape and an ability to grow in adherent culture);
(iii) expresses at least one VLMC marker selected from the group consisting of DCN, LUM, COL1A1, MBP, PRPX1, and MMP2 (e.g., one, two, three, four, five, or all six VLMC markers); and
(iv) expresses at least one additional marker selected from the group consisting of APOE, RSG4, PDGFRA, PDGFRB, NG2, A2B5, CNP, CSPG4, NNAT, CD9, CD146, IGFBP2, MYC, MYT-1, KCNJ8, and OLIG1 (e.g., one, two, three, four, five, six, seven, eight, nine, ten, eleven, twelve, thirteen, fourteen, fifteen, or sixteen of the listed markers).

In embodiments, the VLMCs express DCN, LUM, COL1A1, MBP, PRPX1, and MMP2.

In embodiments, the VLMCs secrete IL-1β upon inflammatory stimulation. In embodiments, inflammatory stimulation comprises culture with LPS.

In another aspect, the disclosure pertains to cultured populations of human VLMCs. For example, in embodiments, the disclosure provides a cultured cell population comprising at least 1×10⁶ (e.g., 1×10⁷, 1×10⁸, 1×10⁹, 1×10¹⁰, or more) of the human VLMCs described herein. The VLMCs are characterized by the morphological features, functional features, and gene/marker expression profiles described herein. In embodiments, the cultured cell population is in suspension culture. In embodiments, the cultured cell population is in adherent culture.

The present invention is further illustrated by the following examples, which should not be construed as further limiting. The contents of figures and all references, patents, and published patent applications cited throughout this application are expressly incorporated herein by reference.

### EXAMPLES

### Example 1: Protocol Development for the Generation of Stem Cell Derived Vascular Leptomeningeal Cells

A stepwise differentiation protocol for generation of vascular leptomeningeal cells (VLMCs) was developed that can guide human pluripotent stem cells to differentiate into cells expressing LUM, DCN, COL1A1, and PDGFRA after 40 days in culture. Differentiated cells also express other vascular leptomeningeal cells genes including APOE, RSG4, and MMP2. This cell type that is derived from ectoderm germ layer also expresses genes such as OLIG2, SOX10, and NG2 that are shared with glial cells and pericyte genes such as CD146, CD44, and PDGFRB (Vanlandewijck et al. (2018) Nature 554:475-480; Zeisel et al. (2018) Cell 174:999-1014; Marques et al. (2018) Dev Cell 46:504-517). After treating the cells with stage 1 pre-oligodendrocyte progenitor cell media (set forth in Table 1) for 3 days and stage 2 oligodendrocyte progenitor cell (OPC) media (set forth in Table 2) for another 3 days, cells were treated with stage 3 media for six days, followed by 28 days in stage 4 media during which cells were split weekly to maintain confluency of culture.

VLMC differentiation recipes were developed as a byproduct of thorough investigation of effects of various agonist and antagonists (effectors) on differentiation of oligodendrocyte progenitor cells using HD-DoE method. These effectors were chosen based on available literature on developmental biology, differentiation of stem cells as well as mouse and human single cell RNA-seq data from oligodendrocytes at the time.

These experiments led to the generation of stage 3 recipe (Table 3) and stage 4 recipe (Table 4).

TABLE 3
Validated factors in stage 3 recipe

| Effectors | Role | Concentration | |
|---|---|---|---|
| FGF-2 | FGFR pathway activator | 10 | ng/ml |
| TTNPB | RA pathway agonist | 50 | nM |
| CHIR99021 | WNT pathway agonist | 1 | μM |
| DBZ | NOTCH pathway inhibitor | 100 | nM |
| PDGF-AA | PDGFR pathway agonist | 10 | ng/ml |
| IGF-1 | IGF-1 pathway agonist | 10 | ng/ml |
| Linoleic Acid | Polyunsaturated fatty acid | 100 | μM |

TABLE 4
Validated factors in stage 4 recipe

| Effectors | Role | Concentration | |
|---|---|---|---|
| NT-3 | TrkC pathway agonist | 10 | ng/ml |
| T3 | THR pathway agonist | 100 | nM |
| cAMP | PKA pathway agonist | 1 | μM |
| DBZ | NOTCH pathway inhibitor | 100 | nM |
| Propionate | Short chain fatty acid | 100 | nM |

To engineer the recipe of stage 3 of differentiation, cells were first cultured in stage 1 and stage 2 media (described in detail in US Publications 2022/0315891 and 2024/0043798). Then, 48 or 96 different combinations of effectors generated using HD-DoE compression through D-optimality were robotically prepared. The effector combinations were prepared in a basal media and were subsequently added to the cells, which were then allowed to differentiate. 3 days later RNA extraction was performed, and gene expression was obtained using quantitative PCR analysis. The data was normalized and modeled using partial least squares regression analysis to the effector design, resulting in the generation of gene-specific models, which after model tuning for maximal predictive power, provided explanation of the effectors ability to control the expression of individual genes, combinatorically, and individually. Solutions within the tested space were then further explored to address desirability.

At this point of differentiation, optimal conditions were studied for maximal expression of SOX10, OLIG1, and NKX2-2 while expression of PDGFRA is at a high level, to positively regulate differentiation of early oligodendrocyte progenitors towards committed PDGFRA⁺ progenitor cells (Emery and Lu (2015) Cold Spring Harb Perspect Biol. 7 (9): a020461; Perlman et al. (2020) Glia 68:1291-1303; Goldman and Kuypers (2015) Development 142:3983-95). SOX10 is one of the main transcription factors of oligodendrocytes, along with OLIG2 and NKX2-2 that has shown the potential to commit the stem cells to oligodendrocytes in reprogramming efforts (Wang et al. (2014) Proc Natl Acad Sci USA 111: E2885-94; García-León et al. (2018) Stem Cell Reports 10:655-672). As it is shown by single cell RNA-seq data, these progenitors are multipotential with the possibility of differentiation towards VLMCs, OPCs, or astrocyte progenitor cells (Marques et al. (2018) Dev Cell 46:504-517; Fu et al. (2021) Cell Rep. 34:108788).

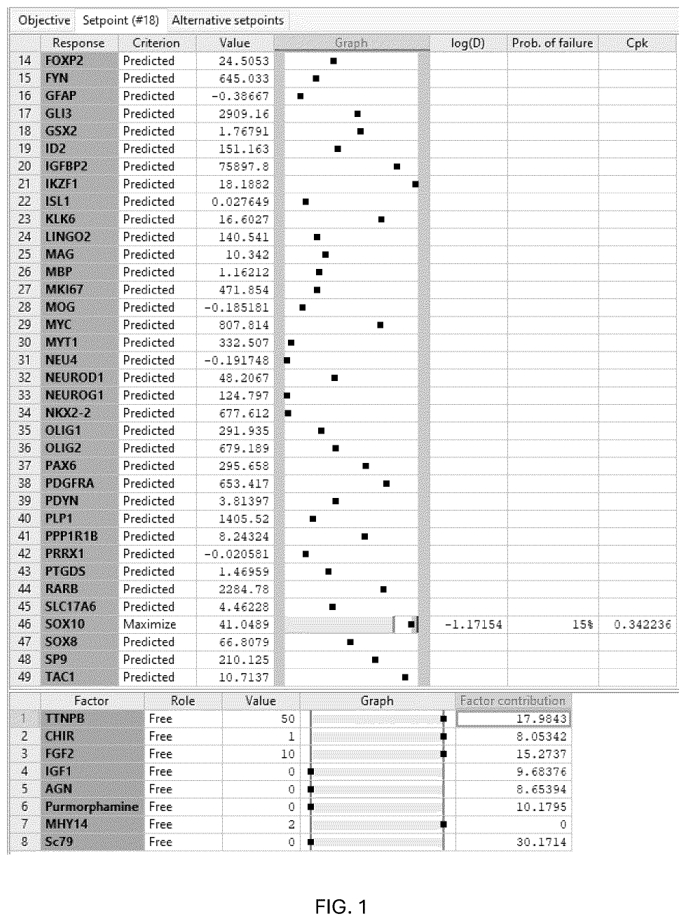

In one 8-factor modeling experiment, the individual and combinatorial effect of CHIR99021 (a WNT pathway agonist), AGN193109 (an RA pathway antagonist), FGF-2 (FGFR pathway agonist), Purmorphamine (a SHH pathway agonist), TTNPB (an RA pathway agonist), IGF-1 (an IGF-1 pathway agonist), MHY1485 (a mTOR pathway agonist), and Sc79 (an Akt pathway agonist), on further differentiation of cells were tested. This experiment demonstrated the potential to regulate pre-OPCs to express SOX10, OLIG2, OLIG1, NKX2-2, and PDGFRA. When the model was optimized for maximum expression of SOX10 at 40, multiple factors showed positive regulatory effect including TTNPB with highest factor contribution at 18, CHIR99021 and FGF-2 with factor contribution of 8 and 15, respectively (FIG. 1). Sc79 had the highest negative impact on its expression with factor contribution of 30. Within the specifications of attaining 80% maximal expression of SOX10, this complex media composition had a Cpk value (process capability index) of 0.34, with a corresponding to a 15% risk of failure. At maximal expression of SOX10, other desired genes such as IGFBP2 and MYC were also upregulated, while neuronal genes NEUROD1 and NEUROG1 were minimally expressed.

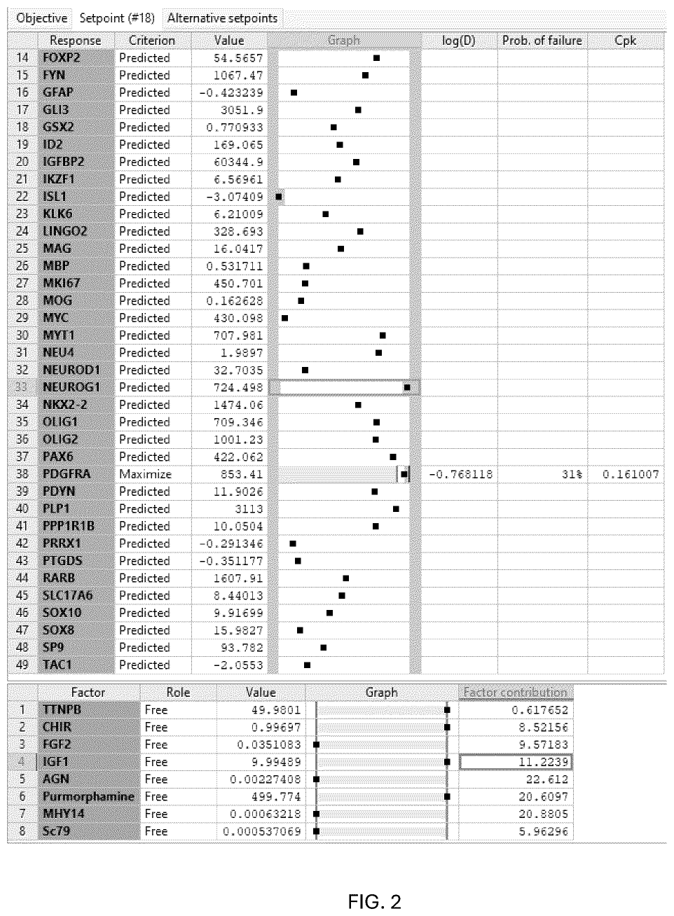

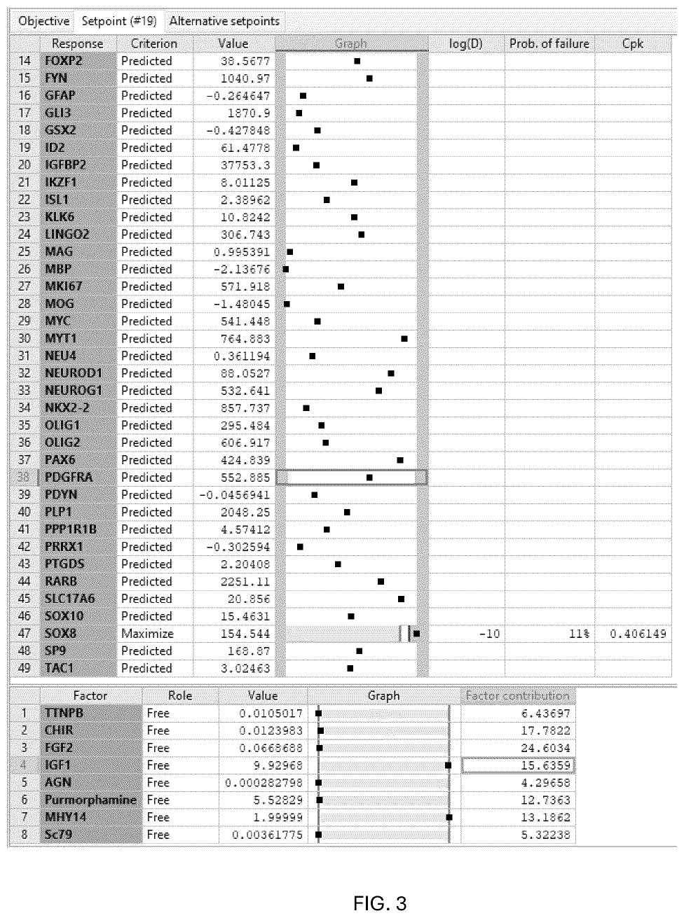

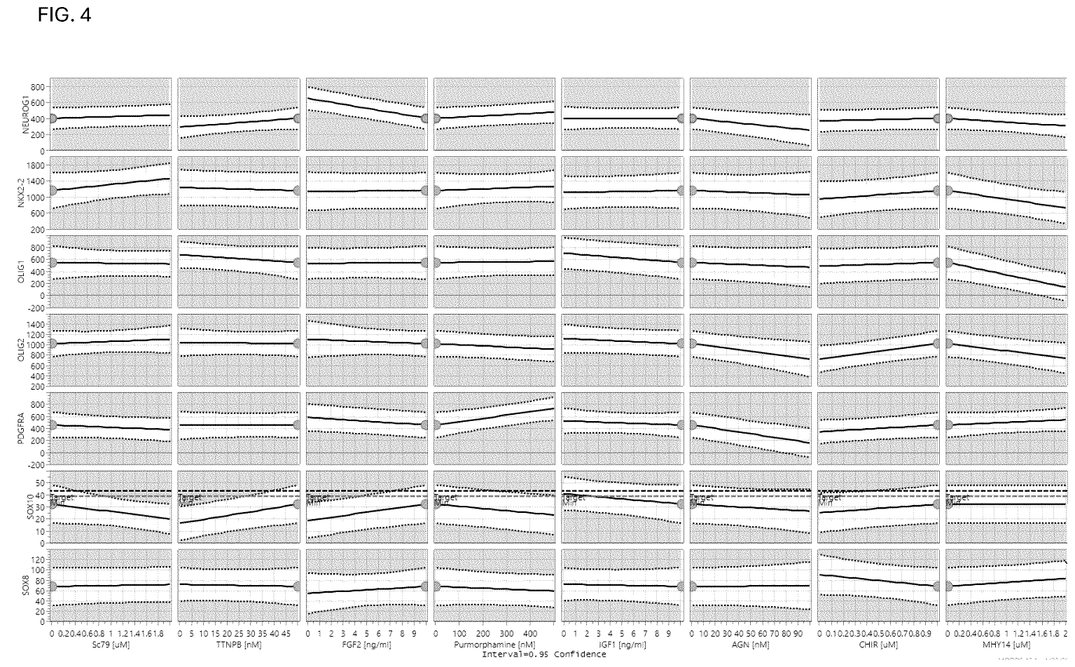

This model was also optimized for maximum expression of PDGFRA at 853 and it was observed that, similar to optimization of SOX10, TTNPB, and CHIR99021 had a positive impact on its regulation with factor contribution of 0.6 and 8. The highest positive contribution was from Purmorphamine with factor contribution of 21 and IGF-1 with factor contribution of 11. MHY1485 and AGN193109 had the highest negative impact with factor contribution of 21 and 23, respectively (FIG. 2). Within the specifications of attaining 80% maximal expression of PDGFRA, this complex media composition had a Cpk value (process capability index) of 0.16, corresponding to a 31% risk of failure. Combinatorial effect of factors was also investigated when experiment was optimized for maximal expression of SOX8, a transcription factor expressed prior to SOX10 which plays a role in differentiation and myelination of oligodendrocytes (Turnescu et al. (2018) Glia 66:279-294) and two factors with positive effect were identified: IGF-1 and MHY1485 with factor contribution of 16 and 13, respectively (FIG. 3). Considering the positive effect of IGF-1 on expression of both PDGFRA and SOX8 and to better understand its effect on our target genes, we used dynamic profile analysis to assess the expression level of genes when model is optimized for SOX10, and IGF-1 is also added to the previously identified upregulating factors. When the model was optimized, expression levels were observed of NKX2-2 at 1200, OLIG1 at 550, OLIG2 at 1000, PDGFRA at 500, SOX8 at 70, and SOX10 at 35 (FIG. 4).

According to the analysis, addition of IGF-1 to the suggested recipe decreased the expression level of SOX10 from 40 to 35, which was still close to predicted range of maximal expression of this gene, therefore three factors, TTNPB, FGF-2, CHIR99021, along with IGF-1 were chosen as candidate factors of stage 3 recipe.

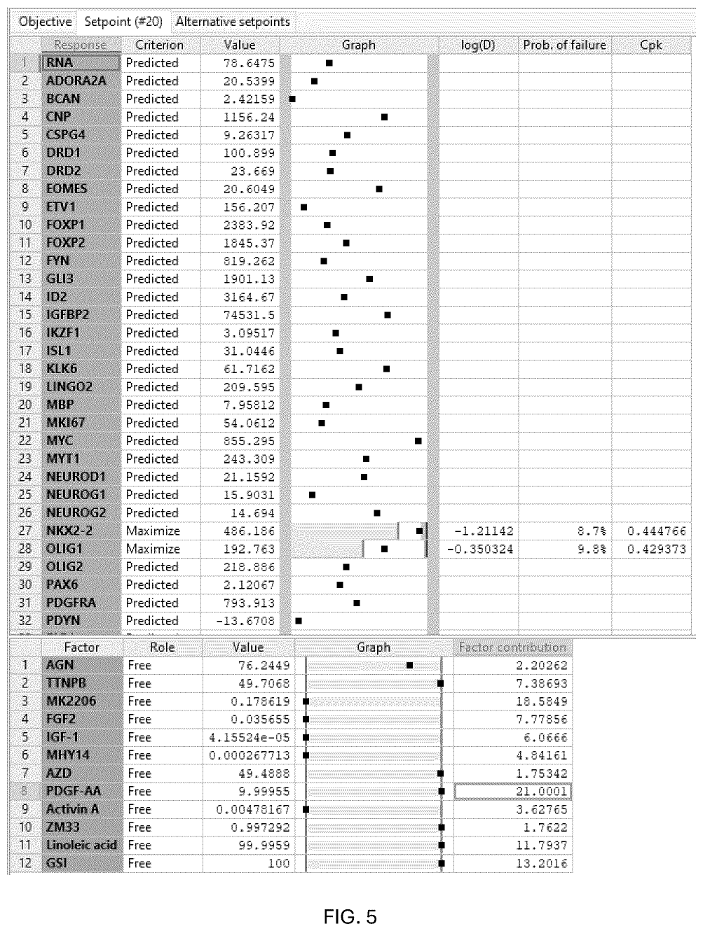

Another 12-factor modeling experiment was performed to study the effect of additional factors on expression level of target genes at stage 3. In this experiment, Activin A, Linoleic acid (a polyunsaturated fatty acid), DBZ (GSI-XX) (a NOTCH pathway inhibitor), ZM336372 (a c-Raf inhibitor), AZD3147 (a mTOR pathway inhibitor), PDGF-AA (a PDGF pathway agonist) and MK2206 (an Akt pathway inhibitor), along with AGN193109, TTNPB, FGF2, IGF-1, and MHY1485, were tested. According to literature, proteins such as FGF-2, IGF-1, and PDGF-AA play an important role in oligodendrocyte differentiation (Goldman and Kuypers (2015) Development 142:3983-95). Models were optimized for maximal expression of OLIG1 with expression level of 192 and NKX2-2 with expression level of 486 simultaneously. Three factors were identified with factor contributions larger than 10, including PDGF-AA, linoleic acid and DBZ with factor contributions of 21, 12 and 13, respectively, that had a positive effect on upregulation of both genes (FIG. 5). MK2206 with factor contribution of 19 had the highest negative impact on expression of these genes. Within the specifications of attaining 80% maximal expression of OLIG1 and NKX2-2, this complex media composition had a Cpk value (process capability index) of 0.44 and 0.42, with a corresponding 8.7% and 9.8% risk of failure for target genes, respectively. To confirm the impact of identified factors on a bigger scale, the expression profile of a larger group of genes defining late oligodendrocyte progenitor population (Perlman et al. (2020) Glia 68:1291-1303; Goldman and Kuypers (2015) Development 142:3983-95; van Tilborg et al. (2018) Glia 66:221-238) was investigated. At maximal expression of OLIG1 and NKX2-2, other desired genes such as CNP, IGFBP2, MYC, and MYT-1 were highly expressed, while neuronal genes, NEUROD1, NEUROG1, and NEUROG2 had expression levels less than 30.

Therefore, considering these models, a candidate recipe for stage 3 consisting of FGF2, TTNPB, CHIR99021, IGF-1, PDGF-AA, linoleic acid, and DBZ was made. This recipe was selected to maximize differentiation of cells, as these agents relate to robust and elevated expression of SOX10, SOX8, PDGFRA, OLIG2, and NKX2-2. This recipe was further validated by immunocytochemistry, qPCR, and flow cytometry assays.

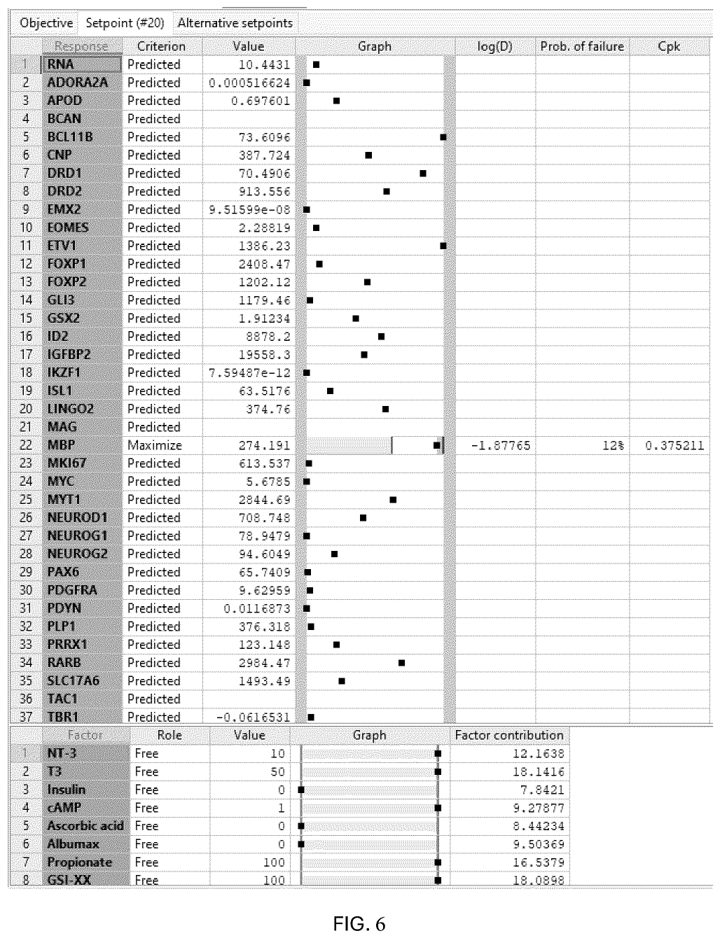

Next, in further experiments, the maximal expression of late oligodendrocyte progenitor genes, such as MBP (Perlman et al. (2020) Glia 68:1291-1303; Goldman and Kuypers (2015) Development 142:3983-95), was focused on. In one 8-factor model, individual and combinatorial effects of NT-3 (a Trk C pathway agonist), T3 (a thyroid hormone receptor agonist), Insulin, CAMP (a PKC pathway agonist), Ascorbic acid, Albumax (a lipid-rich bovine serum albumin), propionate (a short chain fatty acid), and DBZ (a NOTCH pathway inhibitor) were tested. When optimized for MBP at 274, DBZ and T3 showed the highest positive impact both with factor contribution of 18.1. NT-3 and propionate were also positive with factor contribution of 12.1 and 16.5, respectively. cAMP with factor contribution of 9.2 was the fifth identified compound upregulating expression level of selected gene (FIG. 6). Within the specifications of attaining 80% maximal expression of MBP, this complex media composition had a Cpk value (process capability index) of 0.37, corresponding to a 12% risk of failure.

Therefore, considering the models, a candidate recipe for stage 4 consisting of NT-3, T3, CAMP, propionate, and DBZ was made. This recipe was selected to maximize differentiation of cells, as these agents relate to robust and elevated expression of MBP. This recipe was further validated by immunocytochemistry assay.

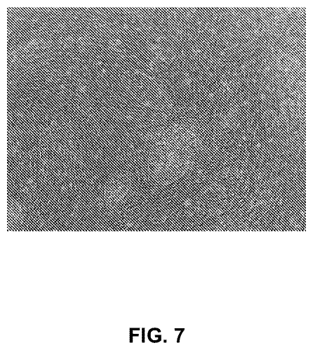

As part of the validation studies to investigate candidate recipes for stage 3 and stage 4 for glial differentiation, this recipe was tested on cells that were treated with VLMC stage 3 recipe for six days. It was observed that this recipe can in fact upregulate PDGFRa and NG2 markers over a 28-day period and cells start expressing VLMC specific markers such as LUM and DCN. As cells were differentiating, a specific fibroblast-like morphology started to form as well (FIG. 7).

### Example 2: Validation of Stem Cell Derived Vascular Leptomeningeal Cells

For validation of developed recipes, the expression of genes such as OLIG1/2, SOX10, PDGFRA, FBLN1, LUM, DCN, CNP, and CSPG4 was assessed by RNA sequencing, and their protein expression by immunocytochemistry and flow cytometry assays. Some of these markers are commonly known to be expressed in oligodendrocyte progenitors and newly formed oligodendrocytes (Marques et al. (2018) Dev Cell 46:504-517). However, published single cell RNA-seq studies done on human and rodent samples in recent years (Vanlandewijck et al. (2018) Nature 554:475-480; Zeisel et al. (2018) Cell 174:999-1014) show oligodendrocyte progenitors and VLMCs share many genes. It is also shown CD146, KCNJ8, and PDGFRB, two known genes of pericyte cells are also present in VLMCs (Smyth et al. (2018) J Chem Neuroanat. 92:48-60). Comparison of the gene profile of generated VLMCs with pre-oligodendrocyte (pre-OL) cells made it possible to better understand the differences between the two populations and helped confirm the identity of generated cells.

### Flow Cytometry Validation of Stem Cell Derived Vascular Leptomeningeal Cells

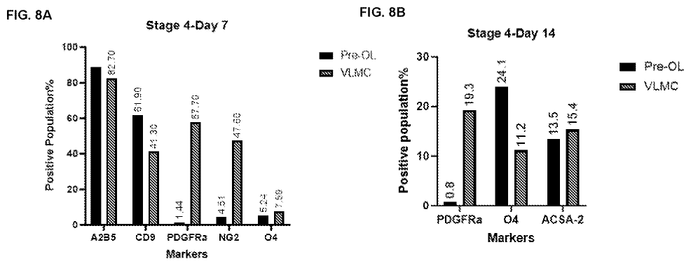

As the first step, to understand the differences in marker expression of generated cells after treatment with developed stage 3 and stage 4 recipes and pre-OL cells, flow cytometry assay was used and percentage of positive population for each marker was obtained. At the end of stage 2, cells were treated with one of two sets of recipes, a validated oligodendrocyte differentiation recipe set and the VLMC developed recipe set (stage 3 and 4) as described in Example 1. Glial markers A2B5, CD9, PDGFRa, NG2, and 04 were measured on day 7 of stage 4 treatment (FIGS. 8A-8B). As shown at FIG. 8A, A2B5 was expressed at over 80% of cells in both populations and 04 was expressed at less than 10% of cells in both. However, distinct differences were observed between the VLMC recipe set and the pre-OL recipe set for other markers. The VLMC recipe led to significantly higher expression of PDGFRa and NG2 markers with 58% and 48% positive population for each, respectively, while the pre-OL recipe set led to higher expression of CD9 that was detected at 62% of population. After treatment with stage 4 media for an additional week (day 14 of stage 4), expression of PDGFRa, 04, and ACSA-2 markers was measured in both populations. As shown in FIG. 8B, VLMCs still had higher expression of PDGFRa at 19% while, pre-OL population didn't express any PDGFRa at this point of differentiation. However, expression of 04 increased from 5% to 24% in pre-OL cells while it was less than half of that in VLMCs. The astrocyte specific marker ACSA-2 is present at both cell cultures at around 15%.

### Bulk RNA-Seq Validation of Stem Cell Derived Vascular Leptomeningeal Cells

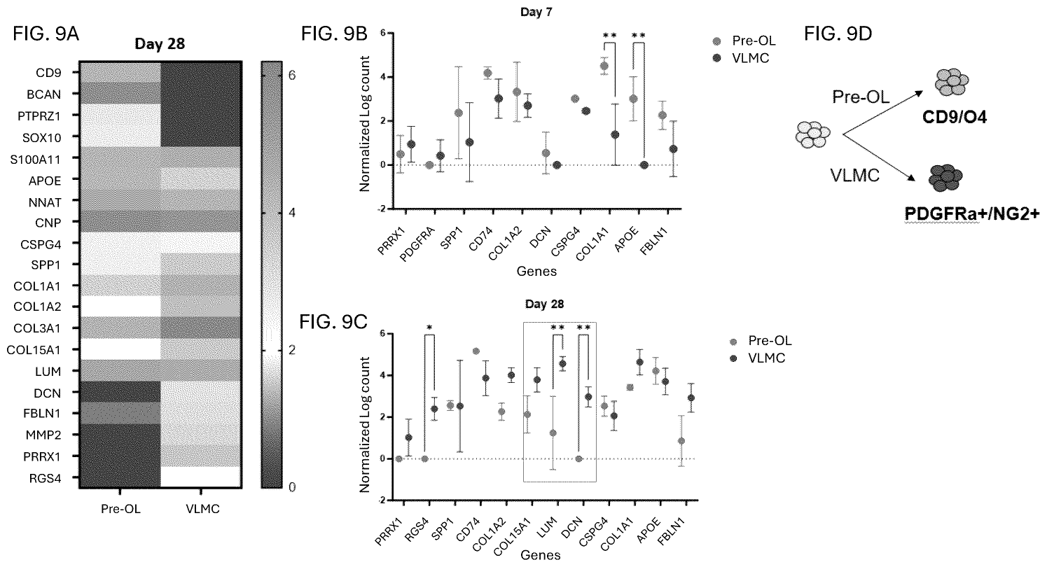

Next, to understand the gene profile of the cell population generated by the recipes described in Example 1 at a larger scale, three replicates of samples of two timepoints during stage 4 differentiation, day 7 and day 28, were submitted for bulk RNA sequencing. The results, shown in FIGS. 9A-9C, show normalized expression levels of a set of selected genes representative of developing oligodendrocyte population and VLMC population. In FIG. 9A, the heatmap of scaled profile of selected genes on day 28 of stage 4 showed that both cell populations expressed shared glial genes, such as CNP, CSPG4, APOE, and NNAT. However, VLMC genes, such as DCN, MMP2, and PRRX1, were only present in the second population and pre-OL genes, including CD9, BCAN, PTPRZ1, and SOX10, are only present in pre-OL population. It was observed that LUM, a VLMC gene, had a high count in the VLMC population but was also present at a low level in pre-OL population. That could be due to heterogenicity of generated cells. FIG. 9B and FIG. 9C show significant difference in log 2 count of VLMC genes on day 7 and day 14 in pre-OL and VLMC populations. On day 7, expression of COL1A1 and APOE, two genes that are expected to be highly expressed in VLMC population (Vanlandewijck et al. (2018) Nature 554:475-480: Zeisel et al. (2018) Cell 174:999-1014), were significantly higher in pre-OL population. However, as cells further differentiate, their normalized count increased and there was no significant difference between pre-OL and generated VLMCs on day 28. Expression level of specific genes, DCN, LUM, and RSG4 genes, was also increased by day 28 and was significantly higher in generated VLMC population.

The RNA-seq and flow cytometry assay findings indicated a multipotential glial progenitor population was generated by culturing iPSCs in the pre-OL stage 1 and stage 2 recipes and that further culture in the stage 3 and stage 4 recipes described in Example 1 guided the cells to a PDGFRa/NG2 positive population that differentiated to VLMCs expressing DCN, LUM, and COL1A1 over time.

### Immunocytochemistry Validation of Stem Cell Derived Vascular Leptomeningeal Cells

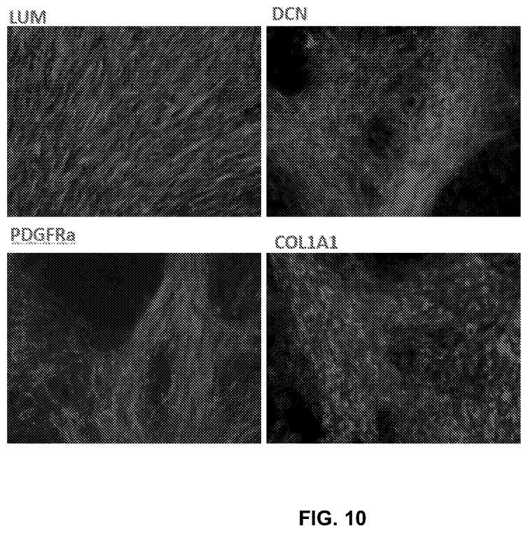

To visualize expression of target markers in cells generated from the VLMC recipes described in Example 1, cells were treated with stage 1 differentiation media for 3 days, stage 2 differentiation media for 3 days, stage 3 differentiation media for 6 days, and stage 4 differentiation media for 28 days, then cells were fixed, and standard immunocytochemistry assays were performed. For staining the cells, VLMC specific markers, including DCN, LUM, and COL1A1, were used, as well as a shared marker with glial cells, PDGFRa. As shown in FIG. 10, four markers were highly expressed in cells, supporting the ability of the developed recipes as a stage-wise differentiation media in directing the cells towards vascular leptomeningeal identity.

### Example 3: Functional Validation of Stem Cell Derived Vascular Leptomeningeal Cells

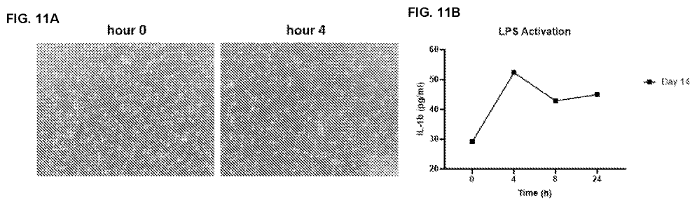

One role of leptomeningeal cells in brain is regulation of inflammatory response of surrounding cells in blood brain barrier region (Ohe et al. (1996) J Neurochem. 67:964-71; Garabedian et al. (2000) Brain Res Mol Brain Res. 75:259-63). It has been reported that leptomeningeal cells activate astrocytes and microglia by secretion of proinflammatory cytokines such as IL-1β and TNF-α (Garabedian et al. (2000) Brain Res Mol Brain Res. 75:259-63; Wu et al. (2005) J Neuroimmunol. 167:90-98). Therefore, to validate the functional response of generated VLMCs to inflammation, an ELISA assay was used to measure secretion of IL-1β when cells were stimulated by lipopolysaccharide (LPS) for up to 24 hours. On day 14 of stage 4 of differentiation, LPS was added to cultures at concentration of 10 ng/ml and the supernatants of 4 wells were collected every 4 hours. An ELISA assay was performed, and results were analyzed following manufacturer's instructions. As shown in FIG. 11, after treatment of cells with LPS for 4 hours, there is a spike in secretion of IL-1β, going from 30 μg/ml to 52 μg/ml, and as time goes on secretion of IL-1β is maintained around 40 μg/ml. These results confirm the capability of the generated cells in responding to inflammatory factors in their environment.

### Example 4: Generation and Validation of Stem Cell Derived Vascular Leptomeningeal Cells in Suspension Culture

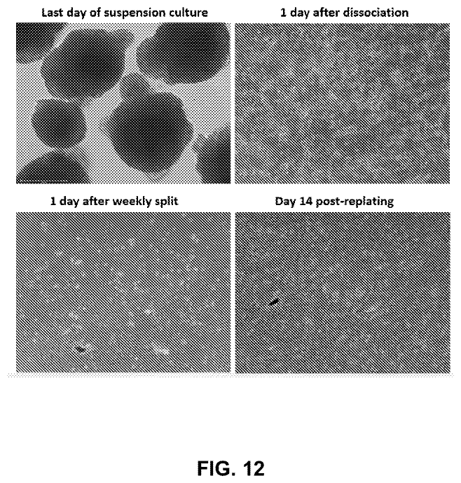

The differentiation protocol described in Example 1 was validated in a suspension culture by following the differentiation protocol from stages 1 to 3 and then plating differentiated size-controlled aggregates onto coated plates for the duration of stage 4. To achieve a homogeneous population, during stage 4 treatment cells were replated every 7 days at low seeding density (FIG. 12). Generated cells were validated by ICC, Flow cytometry, and qPCR on day 28 of stage 4. Samples of day 14 of stage 4 were used for bulk RNA-seq analysis.

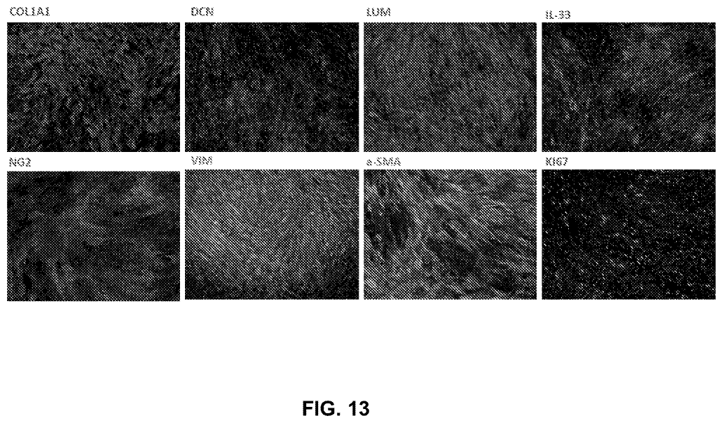

As shown in FIG. 13, generated cells in suspension culture express COL1A1, DCN, LUM, and NG2, which is in alignment with the results of adherent culture. Fixed cells were also stained with additional markers: IL-33, VIM, and a-SMA known to be expressed in VLMCs (Smyth et al. (2018) J Chem Neuroanat. 92:48-60). ICC images confirmed generated cells are VLMCs.

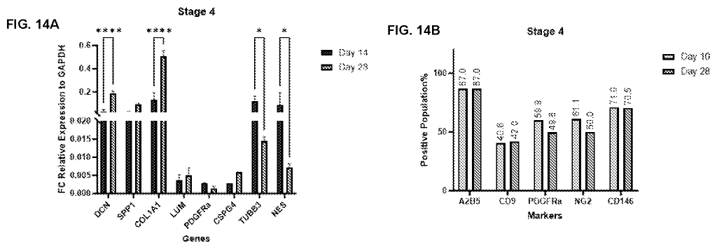

FIG. 14A shows a bar plot depicting relative gene expression of cells on day 14 and 28 of stage 4 of differentiation evaluated by quantitative PCR assay. As cells matured to VLMCs, expression levels of DCN and COL1A1 significantly increased while expression levels of neuronal genes NES and TUBB3 significantly decreased. LUM, PDGFRA, and CSPG4 were expressed in both timepoints. FIG. 14B shows the result of the flow cytometry assay on day 10 and 28 of stage 4 differentiation. The glial surface markers A2B5, CD9, PDGFRA, NG2, and the pericyte marker CD146, which are also expressed in the VLMC population, were measured. It was observed that cultures maintained a consistent expression of A2B5 at 87%, CD9 at around 40%, CD146 at 71% and PDGFRA, and NG2 at 50 to 60% of cells during the terminal differentiation.

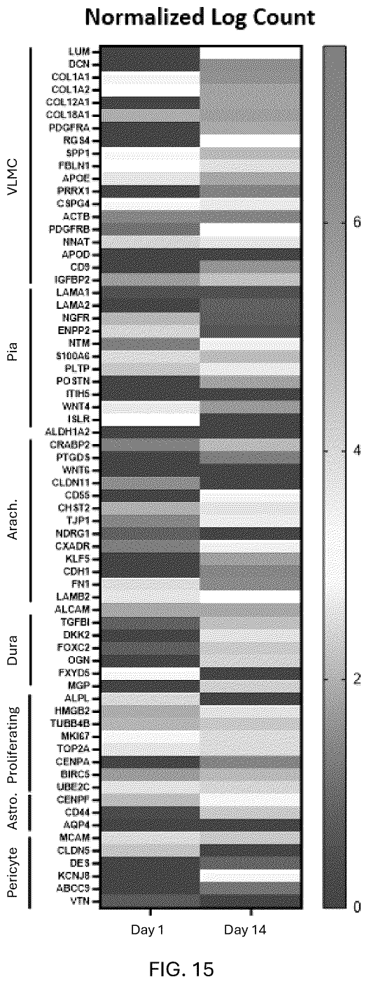

Normalized log count of differentiating cells at day 1 and day 14 of stage 4 was measured by bulk RNA sequencing. FIG. 15 shows the heatmap of selected genes of VLMC, three layers of meninges, pericyte, astrocyte, and proliferating cells. It was observed that many genes of VLMC and meninges were present in generated cell population, while astrocyte and pericyte markers were absent, which confirms the high purity of culture as was observed in morphology of cultured cells (FIG. 12).

### Example 5: Validation of Stem Cell Derived Vascular Leptomeningeal Cells Differentiation Protocol Using a Second iPSC Line

The differentiation protocol described in Example 1 was validated on a second iPSC line (Reprocell StemRNA Human iPSC 771-3G Cat #RCRP005N) and ICC and flow cytometry were performed on cells at day 40 of differentiation to confirm successful differentiation to VLMCs.

iPSCs were adapted to E8 Flex media and VTN coating system and single cell plated for differentiation. After treating the cells in stage 1, 2, 3, and 4 differentiation media followed by weekly splitting of cells in stage 4 media, cultures were either fixed for immunocytochemistry or live cells were harvested for flow cytometry assay.

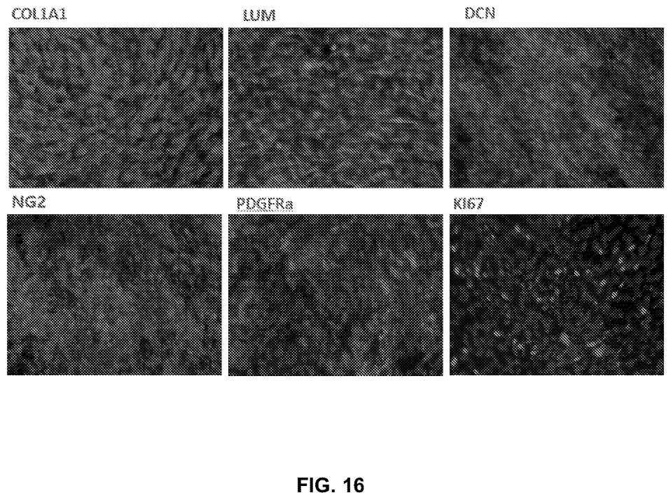

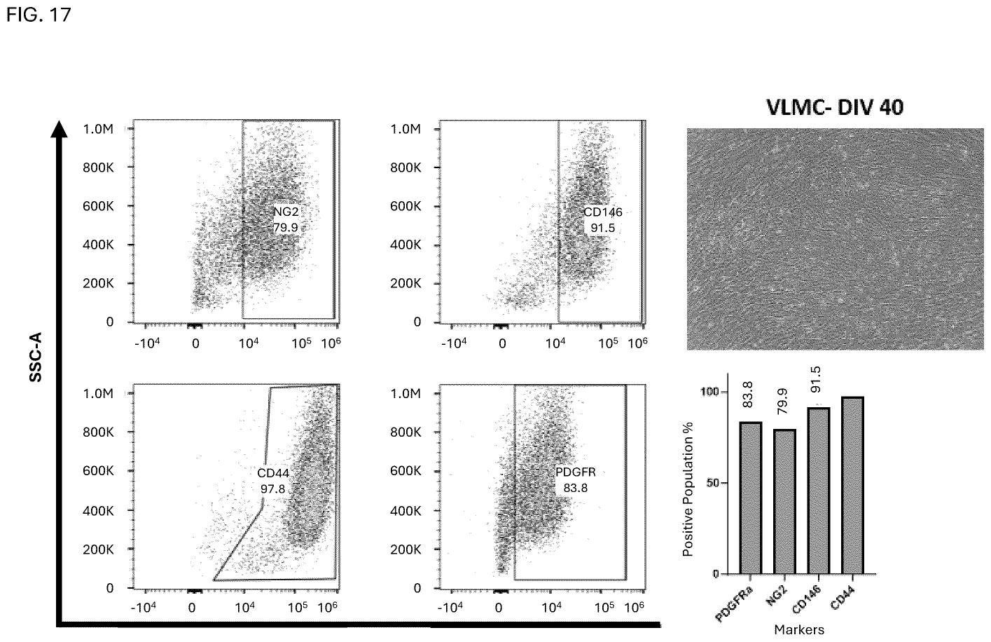

FIG. 16 shows fluorescent images of cells stained by target markers COL1A1, DCN, LUM, PDGFRa, and NG2 and the proliferation marker KI67. As expected, the majority of cells in the culture highly express VLMC markers, confirming the identity of cells. The KI67 marker was also detected in a minimal population in culture, which shows some cells are still proliferating in culture. In flow cytometry, expressions of the surface markers PDGFRa, NG2, CD146, and CD44 were measured, the results of which are shown in FIG. 17. It was observed that more than 80% of cells expressed all four markers, which showcased the homogeneity of the differentiated cells and the ability to control the fate of cells using the developed recipes. High expression of PDGFRa and NG2 was expected and matched the previous findings. Expression of CD44 was also aligned with the RNA-seq analysis of suspension culture, which showed an increasing trend in normalized count of CD44 gene from day 1 to day 14 of stage 4 (FIG. 15). CD44 has been reported to be expressed in both glial and pericyte cell subtypes (Fu et al. (2021) Cell Rep. 34:108788; Zhu et al. (2022) Bone Res. 10:30) and since the VLMC population shares many genes with both cell types, it was concluded that the flow cytometry result further confirmed the identity of generated cells in vitro.

### EQUIVALENTS

Those skilled in the art will recognize, or be able to ascertain using no more than routine experimentation, many equivalents of the specific embodiments of the invention described herein. Such equivalents are intended to be encompassed by the following claims:

1. A method of generating human vascular leptomeningeal cells (VLMCs) from human oligodendrocyte progenitor cells (OPCs), the method comprising:
culturing human OPCs in a first culture media comprising a fibroblast growth factor receptor (FGFR) pathway agonist, a retinoic acid (RA) pathway agonist, a WNT pathway agonist, a NOTCH pathway antagonist, a platelet-derived growth factor receptor (PDGFR) pathway agonist, and an insulin-like growth factor 1 (IGF-1) pathway agonist such that human glial progenitor cells (GPCs) are generated; and
culturing the human GPCs in a second culture media comprising a tropomyosin receptor kinase C (TrkC) pathway agonist, a thyroid hormone receptor (THR) pathway agonist, a protein kinase A (PKA) pathway agonist, and a NOTCH pathway antagonist such that human VLMCs are generated.

2. The method of claim 1, wherein:
(i) the human GPCs are generated after six days of culture of the human OPCs in the first culture media; and/or
(ii) the human VLMCs are generated after 28 days of culture of the human GPCs in the second culture media.

3. (canceled)

4. The method of claim 1, wherein:
(i) the human GPCs express one or more markers selected from the group consisting of PDGFRA, OLIG2, SOX 10, SOX8, NKX2-2, and NG2;
(ii) the human VLMCs express one or more markers selected from the group consisting of DCN, LUM, COL1A1, MBP, PRPX1, and MMP2; and/or
(iii) the human OPCs express one or more markers selected from the group consisting of CD9, BCAN, PTPRZ1, and SOX10.

5. (canceled)

6. The method of claim 4, wherein the human VLMCs also express one or more markers selected from the group consisting of APOE, RSG4, PDGFRA, PDGFRB, NG2, A2B5, CNP, CSPG4, NNAT, CD9, CD146, IGFBP2, MYC, MYT-1, KCNJ8, and OLIG1.

7. (canceled)

8. The method of claim 1, wherein:
(i) the FGFR pathway agonist is selected from the group consisting of FGF2, SUN11602, FGF1, FGF3, FGF4, FGF5, FGF6, FGF8, FGF10, FGF17, FGF19, FGF20, FGF21, FGF22, FGF23, and combinations thereof;
(ii) the RA pathway agonist is selected from the group consisting of TTNPB, retinoic acid (ATRA), EC23, 9-cis-retinoic acid, adapalene, tretinoin, 13-cis retinoic acid (isotretinoin), 4-oxo retinoic acid, WYC-209, DC271, acitretin, arotinoid, AGN205327, LGD1550, Ch55, tazarotene (AGN190168), AM 580, CD2081, BMS 753, tamibarotene, AGN194078, AGN195183, AGN193836, CD2314, CD2019, CD666, C286, BMS 641, AC-55649, AC261066, KCL-286, CD 1530, CD 437, CD2325, BMS 189961, BMS 270394, BMS 961, trifarotene, palovarotene, SR11237, and combinations thereof;
(iii) the WNT pathway agonist is selected from the group consisting of CHIR99021, CHIR98014, SB 216763, SB 415286, LY2090314, 3F8, A 1070722, AR-A 014418, BIO, BIO-acetoxime, AZD1080, alsterpaullone, indirubin-3-oxime, 1-azakenpaullone, kenpaullone, TC-G 24, TWS 119, AT 7519, KY 19382, AZD2858, CHIR98023, 6-BIO, Cazpaullone, Aloisine A, SB41528, SAR502250, Hymenialdisine, Debromohymenialdisine, Dibromocantherelline, Meridianine A, NSC 693868, IM-12, IMID1, IMID2, VP2.51, VP2.54, BIP-135, JGK-263, MMBO, TCS2002, PF-367, BRD0705, BRD3731, AF3581, TDZD 8, NP 031112, NP00111, NP031115, L803, L803-mts, L807-mts, HMK-32, Palinurin, Tricantin, Manzamine A, BTO, VP0.7, VP1.14, VP1.16, VP3.15, VP3.35, SC100, 6j, LCQFGS01, LCQFGS02, 4-3, 4-4, and combinations thereof;
(iv) the NOTCH pathway antagonist is selected from the group consisting of Dibenzazepine (DBZ), GSI-XX, RO4929097, Semagacestat, LY411575, Crenigacestat, DAPT, BMS 906024, Avagacestat, BMS 299897, BMS 433796, BMS 986115, Compound E, Compound W, Compound 18, DFK-167, L-685458, LY900009, MK-0752, MRK 003, MRK 560, PF 3084014, PF 3084014 Hydrobromide, Z-IL-CHO, Begacestat, JLK6, AL101, IMR-1, IMR-1A, CB-103, RIN1, Brontictuzumab, Tarextumab, PF-06650808, FLI-06, Thapsigargin, CAD204520, Tangeretin, Bruceine D, 15D11, Enoticumab, Demcizumab, ABT-165, Navicixizumab, Marimastat, ZLDI-8, and combinations thereof;
(v) the PDGFR pathway agonist is selected from the group consisting of PDGF-AA, PDGF-BB, PDGF-AB, PDGF-CC, PDGF-DD, PBA2-1c, PMP1, PMP2, and combinations thereof;
(vi) the IGF-1 pathway agonist is selected from the group consisting of IGF-1, IGF1-Ado, X10, mecasermin, IGF-2, insulin, Rg5, IGF-1 24-41, IGF-1 30-41, des (1-3) IGF-1, IGF-1 LR3, Demethylasterriquinone B1, and combinations thereof;
(vii) the TrkC pathway agonist is selected from the group consisting of neurotrophin-3 (NT-3), peptidomimetics based on b-turns of NT-3, LM22B 10, GNF 5837, and combinations thereof;
(viii) the THR pathway agonist is selected from the group consisting of T3, T4, Tiratricol, Liothyronine, Octinoxate, 3,5-Diiodothyropropinonic acid, Eprotirome, CO23, Resmetirom, Sobetirome, Sob-AM2, ZTA-261, MB-07811, MB-07344, ALG-055009, and combinations thereof; and/or
(ix) the PKA pathway agonist is selected from the group consisting of cAMP, Dibutyryl-cAMP, 8-Br-CAMP, CAMPS-Sp, CW 008, Forskolin, 8-CPT-CAMP, Adenosine 3′,5′-cyclic Monophosphate, N6-Benzoyl-CAMP, Sodium Salt, Adenosine 3′, 5′-cyclic monophosphate sodium salt monohydrate, (S)-Adenosine, cyclic 3′, 5′-(hydrogenphosphorothioate)triethylammonium, Sp-Adenosine 3′, 5′-cyclic monophosphorothioate triethylammonium salt, Sp-5,6-DCI-cBiMPS, 8-Bromoadenosine 3′, 5′-cyclic Monophosphothioate, Sp-Isomer sodium salt, Adenosine 3′,5′-cyclic Monophosphorothioate, 8-Bromo-CAMP, Sp-Isomer, Sp-8-pCPT-cyclic GMPS Sodium, 8-Bromoadenosine 3′, 5′-cyclic monophosphate, N6-Monobutyryladenosine 3′: 5′-cyclic monophosphate sodium salt, 8-PIP-CAMP, Sp-CAMPS, and combinations thereof.

9. The method of claim 8, wherein:
(i) the FGFR pathway agonist is FGF2;
(ii) the RA pathway agonist is TTNPB;
(iii) the WNT pathway agonist is CHIR99021;
(iv) the NOTCH pathway antagonist is DBZ;
(v) the PDGFR pathway agonist is PDGF-AA;
(vi) the IGF-1 pathway agonist is IGF-1;
(vii) the TrkC pathway agonist is NT-3;
(viii) the THR pathway agonist is T3; and/or
(ix) the PKA pathway agonist is cAMP.

10. The method of claim 9, wherein:
(i) FGF2 is present in the first culture media at a concentration of 1-20 ng/ml;
(ii) TTNPB is present in the first culture media at a concentration of 10-100 nM;
(iii) CHIR99021 is present in the first culture media at a concentration of 0.3-3.0 μM;
(iv) DBZ is present in the first culture media at a concentration of 25-200 nM;
(v) DBZ is present in the second culture media at a concentration of 25-200 nM;
(vi) PDGF-AA is present in the first culture media at a concentration of 2-20 ng/ml;
(vii) IGF-1 is present in the first culture media at a concentration of 2-20 ng/ml;
(viii) NT-3 is present in the second culture media at a concentration of 2-20 ng/ml;
(ix) T3 is present in the second culture media at a concentration of 50-150 nM; and/or
(x) CAMP is present in the second culture media at a concentration of 0.5-2.5 μM.

11-26. (canceled)

27. The method of claim 1, wherein:
(i) the first culture media further comprises a polyunsaturated fatty acid (PFA); and/or
(ii) the second culture media further comprises a short chain fatty acid (SCFA).

28. The method of claim 27, wherein:
(i) the PFA is selected from the group consisting of linoleic acid, a-linoleic acid (ALA), stearidonic acid (SDA), eicosapentaenoic acid (EPA), docosapentaenoic acid (DPA), docosahexaenoic acid (DHA), g-linoleic acid (GLA), dihomo-g-linoleic acid (DGLA), hexadecatrienoic acid (HTA), eicosatrienoic acid (ETE), eicosatetraenoic acid (ETA), heneicosapentaenoic acid (HPA), tetracosapentaenoic acid, tetracosahexaenoic acid, eicosadienoicd acid, arachidonic acid (AA), docosadienoic acid, adrenic acid (AdA), tetracosatetraenoic acid, tetracosapentaenoic acid, conjugated linoleic acid (CLA), conjugated linolenic acid, rumelenic acid, α-parinaric acid, β-parinaric acid, bosseopentaenoic acid, pinolenic acid, sciadonic acid, and combinations thereof; and/or
(ii) the SCFA is selected from the group consisting of propionate (propionic acid), acetate (acetic acid), butyrate (butyric acid), valerate (valeric acid), isobutyrate (isobutyric acid), isovalerate (isovaleric acid), 2-methylbutanoate (2-methylbutyric acid), and combinations thereof.

29. The method of claim 28, wherein:
(i) the PFA is linoleic acid; and/or
(ii) the SCFA is propionate.

30. The method of claim 29, wherein:
(i) linoleic acid is present in the first culture media at a concentration of 25-200 μM; and/or
(ii) propionate is present in the second culture media at a concentration of 50-150 nM.

31-43. (canceled)

44. A culture media for obtaining human GPCs comprising an FGFR pathway agonist, an RA pathway agonist, a WNT pathway agonist, a NOTCH pathway antagonist, a PDGFR pathway agonist, and an IGF-1 pathway agonist.

45. The culture media of claim 44, further comprising a PFA.

46. A culture media for obtaining human VLMCs comprising a TrkC pathway agonist, a THR pathway agonist, a PKA pathway agonist, and a NOTCH pathway antagonist.

47. The culture media of claim 46, further comprising a SCFA.

48-57. (canceled)

58. A method of generating human VLMCs from human PSCs, the method comprising:
(a) culturing human PSCs in a first culture media comprising an RA pathway agonist, an AKT pathway agonist, a mammalian target of rapamycin (mTOR) pathway agonist, a WNT pathway antagonist agonist, a sonic hedgehog (SHH) pathway agonist, a bone morphogenetic protein (BMP) pathway antagonist, and a protein kinase C (PKC) pathway antagonist such that human pre-OPCs are generated;
(b) culturing the human pre-OPCs in a second culture media comprising a FGFR pathway agonist, a SHH pathway agonist, an AKT pathway antagonist, an AKT pathway agonist, and an mTOR pathway antagonist such that human OPCs are generated;
(c) culturing the human OPCs in a third culture media comprising a FGFR pathway agonist, an RA pathway agonist, a WNT pathway agonist, a NOTCH pathway antagonist, a PDGFR pathway agonist, and an IGF-1 pathway agonist such that human GPCs are generated; and
(d) culturing the human GPCs in a fourth culture media comprising a TrkC pathway agonist, a THR pathway agonist, a PKA pathway agonist, and a NOTCH pathway antagonist such that human VLMCs are generated.
